(19) United States
(12) Patent Application Publication
(10) Pub. No.: US 2025/0163440 A1
(43) Pub. Date: May 22, 2025

(54) COMPOSITIONS AND METHODS FOR IMPROVED RHIZOBIUM-MEDIATED PLANT TRANSFORMATION

(71) Applicant: Board of Regents for the Oklahoma Agricultural and Mechanical Colleges, Stillwater, OK (US)

(72) Inventors: Vidhyavathi Raman, St. Paul, MN (US); Clemencia Rojas, Lincoln, NE (US); Kirankumar S. Mysore, Ardmore, OK (US)

(21) Appl. No.: 18/840,628

(22) PCT Filed: Feb. 22, 2023
(86) PCT No.: PCT/US23/62983

§ 371 Date: Aug. 22, 2024

Related U.S. Application Data
(60) Provisional application No. 63/312,521, filed on Feb. 22, 2022.

(51) Int. Cl. C12N 15/82; C07K 14/21; C07K 14/415; C12N 1/20; C12N 9/22; C12N 15/11; C12R 1/01

(52) U.S. Cl. CPC C12N 15/8205; C07K 14/21; C07K 14/415; C12N 1/20; C12N 9/22; C12N 15/11; C12N 15/8216; C12N 2310/20; C12R 2001/01

(57) ABSTRACT

The present disclosure provides compositions for improved Rhizobium-mediated transformation in transformation of recalcitrant plants, and methods of use thereof. Also provided are compositions and methods for Rhizobium-dependent delivery of heterologous proteins directly to plant cells.

### REFERENCE TO RELATED APPLICATIONS

This application claims benefit under 35 USC § 119 (e) of U.S. provisional Application No. 63/312,521, filed Feb. 22, 2022. The entire contents of the above-referenced patent application(s) are hereby expressly incorporated herein by reference.

### STATEMENT REGARDING FEDERALLY SPONSORED RESEARCH OR DEVELOPMENT

This invention was made with government support under Grant Number IOS-1725122 awarded by the National Science Foundation. The government has certain rights in the invention.

### BACKGROUND

Agrobacterium-mediated transformation is an important and useful method in modern plant biotechnology and plant research. Agrobacterium sp., including A. tumefaciens, are soil borne phytopathogenic bacteria that cause crown gall disease in plants. This disease is a manifestation of the transfer, integration, and expression of oncogenes on a specific region of the transferred DNA (T-DNA) in susceptible hosts. The property of T-DNA transfer has been widely adopted by the field of plant molecular biology to generate transgenic plants containing genes of interest for research purposes or for commercial applications. However, Agrobacterium-mediated transformation has been greatly limited in a number of plant species due to their resistance to infection by Agrobacterium. Efficiency of Agrobacterium-mediated transformation in these species is low at best, due in part to the elicitation of host defense responses. These limitations are typically addressed by generating transgenic plants that express microbial effector proteins that suppress the plant's innate immunity. While this approach results in improving transient transformation in recalcitrant species, these methods involve generation of transgenic plants that conditionally express microbial proteins and do not reflect the true physiological status of the wild-type plants. Improved methods of Rhizobium-mediated transformation generally to increase transformation efficiencies and that do not require generation of transgenic plants that conditionally express microbial effector proteins would therefore represent a significant advancement in the art. Accordingly, the present disclosure includes compositions and methods for improving Agrobacterium-mediated transformation in recalcitrant species and improving transformation efficiencies in general. Additionally, compositions and methods for Agrobacterium-dependent delivery of heterologous proteins directly to plant cells are also disclosed herein.

### BRIEF DESCRIPTION OF THE DRAWINGS

The following drawings form part of the present specification and are included to further demonstrate certain non-limiting aspects of the present disclosure. The disclosure may be better understood by reference to one or more of these drawings in combination with the detailed description of specific embodiments presented herein.

FIG. 1 shows the development and results of secretion assays using Pseudomonas type III secretion system (“T3SS”) when expressed in A. tumefaciens. Panel A shows a schematic of engineering A. tumefaciens to deliver proteins through T3SS for a secretion assay. A. tumefaciens strain GV2260 is transformed with P. syringae pv. syringae 61 (Pss61) T3SS (pLN18) and type III effector AvrPto tagged with PhiLOV to express T3SS and AvrPto-PhiLOV. Panel B shows western blot data using a PhiLOV specific antibody. A. tumefaciens strains GV2260, GV2260 (pLN18), and GV2260 (AvrPto-PhiLOV) were included as controls. Panel C shows schematics of engineering A. tumefaciens for in planta visualization. GV2260 is transformed with pLN18 and AvrPto-GFP₁₁ to express T3SS and AvrPto-GFP₁₁. AvrPto-GFP₁₁ can be translocated to plant cells through T3SS when recombinant A. tumefaciens strains are used to infiltrate N. benthamiana leaves. AvrPto-GFP₁₁ is able to complement GFP₁₋₁₀ expressed transiently in the leaves and give functional GFP and fluorescence that can be detected by confocal microscope. Panel D shows N. benthamiana leaves were infiltrated with A. tumefaciens strain GV2260 expressing Pss61 T3SS (pLN18) and type III effector AvrPto tagged with GFP₁₁. GV2260 and GV2260 (AvrPto-GFP₁₁) were used as negative controls. Twenty-four hours prior to infiltration of these strains, GFP₁₋₁₀ was transiently expressed in plants. Confocal microscope was used to visualize GFP fluorescence 48 h post-infiltration (Scale=10 μm).

FIG. 2 shows A. tumefaciens strains heterologously expressing Pss61 type III secretion system and AvrPtoB or HopAO1 significantly increasing stable transformation efficiency in A. thaliana and N. benthamiana. Panel A shows A. thaliana root segments infected with Agrobacterium tumorigenic strain A208 expressing type III effectors AvrPtoB and HopAO1 in combination with or without pLN18. A208 alone and A208 containing pLN18 and vector, pBBR1MCS5 were included as negative controls. Photographs were taken four weeks after Agrobacterium infection. Panel B shows quantification of root tumors. Number of root segments forming tumors were counted from the experiment in FIG. 2A and percentage of root segments forming tumors were calculated. Panel C shows results of leaf disc transformation assay. N. benthamiana leaf discs were infected with tumorigenic strain A348 expressing AvrPtoB and HopAO1 in combination with or without pLN18. A348 alone and A348 containing pLN18 and pBBR1MCS5 were included as negative controls. Ten days after Agrobacterium infection, photographs were taken. Panel D shows quantification of leaf disc tumor assay. Number of leaf discs developing tumors were evaluated from the experiment shown in FIG. 2, Panels B, C, and D. Percentage of root segments forming tumors and fresh weight of tumors were subjected to statistical analysis. Data presented are mean±standard error of three replicates. Bars with different letters are significantly different according to Tukey's post-hoc test (P<0.05).

FIG. 3 shows Agrobacterium expressing T3SS and T3Es modulate stable transformation in wheat. Immature embryos were infected with AGL-1 (pANIC 6B) expressing pLN18 and T3Es AvrPtoB or HopAO1. AGL-1 (pANIC6B) alone and with AvrPtoB or HopAO1 were included as controls. Regenerated callus that are forming shoots were counted. Data presented are mean±standard error of three replicates. Bars with different letters are significantly different according to Tukey's post-hoc test (P<0.05).

FIG. 4 shows A. tumefaciens strains expressing T3SS and AvrPto significantly reduces plant defense genes expression in A. thaliana. Roots of A. thaliana Col-0 plants infected with Agrobacterium tumorigenic strain A208 expressing AvrPto in combination with and without pLN18. Mock infection and A208 alone were included as negative controls. Roots are harvested 2 and 16 h after infection, rinsed with water were used for qRT-PCR. Panel A shows expression levels of FRK1, which were calculated using 2^{-ΔΔCT} method with UBQ10 gene as a house keeping control. Data presented are mean±standard error of three replicates. Bars with different letters are significantly different according to Tukey's post-hoc test (P<0.05). Panel B shows expression levels of NHL10, which were calculated using 2^{-ΔΔCT} method with UBQ10 gene as a house keeping control. Data presented are mean±standard error of three replicates. Bars with different letters are significantly different according to Tukey's post-hoc test (P<0.05).

FIG. 5 shows A. tumefaciens strains heterologously expressing Pss61 type III secretion system and HTA1 significantly increase stable transformation efficiency in A. thaliana and N. benthamiana. Panel A shows results of root tumor assay. A. thaliana root segments were infected with Agrobacterium tumorigenic strain A208 expressing full length HTA1 or truncated HTA1 (t-HTA1) in combination with or without pLN18. A208 alone and A208 containing pLN18 and vector, pBBR1MCS5 were included as negative controls. Photographs were taken four weeks after Agrobacterium infection. Panel B shows quantification of root tumors. Number of root segments forming tumors were counted from the experiment shown in FIG. 5, Panel A and percentage of root segments forming tumors were calculated. Panel C shows result of leaf disc transformation assay. N. benthamiana leaf discs were infected with tumorigenic strain A348 expressing HTA1 or t-HTA1 in combination with or without pLN18. A348 alone and A348 containing pLN18 and pBBR1MCS5 were included as negative controls. Ten days after Agrobacterium infection, photographs were taken. Panel D shows quantification of leaf disc tumor assay. Number of leaf discs developing tumors were evaluated from the experiment shown in FIG. 5, Panels C, B, and D. Percentage of root segments forming tumors and fresh weight of tumors were subjected to statistical analysis. Data presented are mean±standard error of three replicates. Bars with different letters are significantly different according to Tukey's post-hoc test (P<0.05).

FIG. 6 shows A. tumefaciens strains heterologously expressing Pss61 type III secretion system deliver type III effectors into plant cells. Panel A shows in planta visualization of PhiLOV tagged AvrPto. N. benthamiana leaves were infiltrated with A. tumefaciens strain GV2260 expressing Pss61 T3SS (pLN18) and type III effector AvrPto tagged with PhiLOV. GV2260 (AvrPto-PhiLOV) was used as negative control. Confocal microscope was used to visualize PhiLOV fluorescence 48 h post-infiltration (Scale=10 μm). Panel B shows plasma membrane localization of AvrPto. A. tumefaciens strain GV2260 (AvrPto-GFP₁₁, PLN18) infiltrated to N. benthamiana leaves transiently expressing GFP₁₋₁₀. Twenty-four hours prior to infiltration of these strains, GFP₁₋₁₀ was transiently expressed in plants. Confocal microscope was used to visualize fluorescence 48 hours post-infiltration. GFP signals and auto-fluorescence are pseudo-colored to green, and FM4-64 is shown in red (Scale=10 μm). Panel C shows A. tumefaciens strains heterologously expressing Pss61 type III secretion system deliver other type III effectors into plant cells. N. benthamiana leaves were infiltrated with A. tumefaciens strain GV2260 expressing Pss61T3SS (pLN18) and type III effector AvrB and AvrPtoB tagged with GFP₁₁. GV2260 (AvrPtoB-GFP₁₁) and GV2260 (AvrB-GFP₁₁) were used as negative controls. Twenty-four hours prior to infiltration of these strains, GFP₁₋₁₀ was transiently expressed in plants. Confocal microscope was used to visualize GFP fluorescence 48 hours post-infiltration (Scale=20 μm).

FIG. 7 shows the results of infection of N. benthamiana with A. tumefaciens strains heterologously expressing Pss61type III secretion system and AvrPto. Panel A shows transient transformation efficiency. N. benthamiana leaf discs were incubated with Agrobacterium strain GV2260 harboring the GUS reporter gene in pCAMBIA1301 alone or in combination with plasmids encoding type III secretion (pLN18), vector alone (pBBR1MCS4) or AvrPto (pBBR1MCS4::AvrPto); expression of β-glucuronidase (GUS), associated with transient transformation was visualized by histochemical staining after 10 days of Agrobacterium inoculation. Panel B shows stable transformation efficiency. N. benthamiana leaf discs were incubated with tumorigenic strains A348 or A348 expressing type III secretion (pLN18) and AvrPto (pBBR1MCS4::AvrPto). Ten days after infection the number of leaf discs developing tumors (Panel C), fresh weight (Panel D), and dry weight of tumors were quantified (Panel E).

FIG. 8 shows the results of infection of A. thaliana with A. tumefaciens strains heterologously expressing Pss61type III secretion system and AvrPto. Panel A shows transient transformation efficiency, A. thaliana leaves were infiltrated with Agrobacterium strain GV3101 harboring the GUS reporter gene in pCAMBIA1301 alone or in combination with plasmids encoding type III secretion (pLN18), vector alone (pBBR1MCS4) or AvrPto (pBBR1MCS4::AvrPto); expression of β-glucuronidase (GUS), associated with transient transformation was visualized by histochemical staining after 10 days of Agrobacterium inoculation. Panel B shows stable transformation efficiency, A. thaliana root segments were incubated with tumorigenic strains A208 or A208 expressing type III secretion (pLN18) and AvrPto (pBBR1MCS4::AvrPto) and the developing tumors were evaluated after 10 days. Panel C shows data measuring the percentage of root segments forming tumors when root segments were incubated with tumorigenic strains A208 or A208 expressing type III secretion (pLN18) and AvrPto (pBBR1MCS4::AvrPto) and the developing tumors were evaluated after 10 days.

FIG. 9 shows Agrobacterium expressing T3SS and AvrPtoB and HopAO1 modulate stable transformation in wheat. Representative images from various stages of wheat transformation process are shown: immature embryos (Panel A), infected immature embryos on co-cultivation medium (Panel B), callus induction (Panel C), regeneration (Panel D), transgenic wheat plants transferred to soil in the green house (Panel E), GUS-stained leaf (Panel F), and PCR gels of GUS (Panel G, top panel) and Hyg (Panel G, bottom panel) specific primers for confirmation of transgenics.

FIG. 10 shows Vir gene expression of A. tumefaciens strains expressing T3SS and AvrPto. A. tumefaciens tumorigenic strain A208 expressing AvrPto in combination with and without pLN18 were used. A. tumefaciens tumorigenic strain A208 alone and A208 containing pLN18 and vector were included as negative controls. Roots harvested 2 and 16 hours after infection were used for qRT-PCR. Panel A shows the expression level of VirD2, which was calculated using the 2^{-ΔΔCT} method with the recA gene as a housekeeping control. Data presented are mean±standard error of three replicates. Bars with different letters are significantly different according to Tukey's post-hoc test (P<0.05). Panel B shows the expression level of VirE2, which was calculated using the 2^{-ΔΔCT} method with the recA gene as a housekeeping control. Data presented are mean±standard error of three replicates. Bars with different letters are significantly different according to Tukey's post-hoc test (P<0.05). Panel C shows the expression level of VirA, which was calculated using the 2^{-ΔΔCT} method with the recA gene as a housekeeping control. Data presented are mean±standard error of three replicates. Bars with different letters are significantly different according to Tukey's post-hoc test (P<0.05). Panel D shows the expression level of VirB2, which was calculated using the 2^{-ΔΔCT} method with the recA gene as a housekeeping control. Data presented are mean±standard error of three replicates. Bars with different letters are significantly different according to Tukey's post-hoc test (P<0.05).

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2247; and Lewin, Genes IX, Oxford University Press: New York, 2007. The nomenclature for DNA bases as set forth at 37 CFR § 1.822 is used.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, l-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

As used herein, the term “chimeric” refers to the product of the fusion of portions of two or more different polypeptide, polynucleotide, or to a gene expression element produced through the manipulation of known elements or other polynucleotide molecules.

As used herein, “codon-optimized” refers to a polynucleotide sequence that has been modified to exploit the codon usage bias of a particular plant. The modified polynucleotide sequence still encodes the same, or substantially similar, polypeptide as the original sequence but uses codon nucleotide triplets that are found in greater frequency in a particular plant.

As used herein, “CRISPR-associated genes” refers to nucleic acid sequences that encode polypeptide components of clustered regularly interspersed short palindromic repeats (CRISPR)-associated systems (Cas). Examples include, but are not limited to, Cas9, Cpf1 (aka Cas12a), C2c1, C2c2 (aka Cas13a), Cas12b, Cas14, Cas12e, which encode endonucleases from the CRISPR type I and type II systems.

As used herein, “expression” refers to the combination of intracellular processes, including transcription and translation, undergone by a coding DNA molecule such as a structural gene to produce a polypeptide. A plant in accordance with the present disclosure may exhibit altered expression of a gene set forth herein. Such altered expression may include increased expression, decreased expression, or complete absence of expression.

As used herein, “transformation” refers to a process of introducing a DNA sequence or construct (e.g., a vector or expression cassette) into a cell or protoplast in which that exogenous DNA is incorporated into a chromosome or is capable of autonomous replication.

As used herein, “heterologous” refers to a sequence which is not normally present in a given host genome in the genetic context in which the sequence is currently found. In this respect, the sequence may be native to the host genome, but be rearranged with respect to other genetic sequences within the host sequence. The sequence may also be altered, i.e., mutated, with respect to the native regulatory sequence. For example, a regulatory sequence may be heterologous in that it is linked to a different coding sequence relative to the native regulatory sequence.

As used herein, “transformation construct” refers to a chimeric DNA molecule which is designed for introduction into a host genome by genetic transformation. Particular (but non-limiting) transformation constructs will comprise all of the genetic elements necessary to direct the expression of one or more exogenous genes. In particular (but non-limiting) embodiments of the present disclosure, it may be desirable to introduce a transformation construct into a host cell in the form of an expression cassette.

A used herein, “transformed cell” refers to a cell in which the DNA complement has been altered by the introduction of an exogenous DNA molecule into that cell.

As used herein, “single-guide RNA (sgRNA)” refers to a crRNA: tracrRNA fused hybrid single-stranded RNA molecule encoded by a customizable DNA element that, generally, comprises a copy of a spacer sequence which is complementary to the protospacer sequence of the genomic target site, and a binding domain for an associated-Cas endonuclease of the CRISPR complex.

As used herein, “protospacer” refers to a short DNA sequence (12 to 40 bp) that can be targeted for mutation, and/or double-strand break, mediated by enzymatic cleavage with a CRISPR system endonuclease guided by complementary base-pairing with the spacer sequence in the crRNA or sgRNA.

As used herein, “protospacer adjacent motif (PAM)” includes a 3 to 8 bp sequence immediately adjacent to the protospacer sequence in the genomic target site.

As used herein, “microhomology” refers to the presence of the same short sequence (1 to 10 bp) of bases in different polynucleotide molecules.

As used herein, “non-protein-coding RNA (npcRNA)” refers to a non-coding RNA (ncRNA) which is a precursor small non-protein coding RNA, or a fully processed non-protein coding RNA, which are functional RNA molecules that are not translated into a protein.

As used herein, “promoter” refers to a nucleic acid sequence located upstream or 5′ to a translational start codon of an open reading frame (or protein-coding region) of a gene and that is involved in recognition and binding of RNA polymerase I, II, or III and other proteins (trans-acting transcription factors) to initiate transcription.

As used herein, an “expression cassette” refers to a polynucleotide sequence comprising at least a first polynucleotide sequence capable of initiating transcription of an operably linked second polynucleotide sequence and optionally a transcription termination sequence operably linked to the second polynucleotide sequence.

The following is a detailed description of the present disclosure provided to aid those skilled in the art in practicing the present disclosure. Those of ordinary skill in the art may make modifications and variations in the embodiments described herein without departing from the spirit or scope of the present disclosure.

Certain non-limiting embodiments of the present disclosure relate generally to the field of plant molecular biology. More specifically (but not by way of limitation), certain non-limiting embodiments of the present disclosure relate to compositions for improved Agrobacterium-mediated transformation and methods of use thereof. Also provided are compositions and methods for Agrobacterium-dependent delivery of heterologous proteins directly to plant cells.

In one non-limiting embodiment, the present disclosure provides a recombinant Rhizobium cell comprising a bacterial type III secretion system (T3SS), wherein said cell comprises a recombinant DNA construct comprising at least a first nucleic acid sequence operably linked to a T3SS-specific promoter, wherein said first nucleic acid sequence encodes a site-specific DNA modifying enzyme comprising a T3SS signal sequence. In one non-limiting embodiment, the site-specific DNA modifying enzyme is selected from the group consisting of: an endonuclease, a recombinase, a transposase, a deaminase, and a helicase. In another non-limiting embodiment, the site-specific DNA modifying enzyme is a CRISPR endonuclease. In a further non-limiting embodiment, the site-specific DNA modifying enzyme is selected from the group consisting of: SaCas9, SpCas9, StCas9, NmCas9, FnCas9, CjCas9, ScCas9, Cas-CLOVER, xCas9, Cas12a, C2c1, C2c2, Cas13a, Cas12b, Cas14, Cas12k, and Cas12e. In one non-limiting embodiment, the recombinant DNA construct comprises a second nucleic acid sequence operably linked to a T3SS-specific promoter, wherein said second nucleic acid sequence encodes a heterologous protein that functions to suppress innate immunity in plants. In a further non-limiting embodiment, the heterologous protein is a bacterial type III effector protein. In yet a further non-limiting embodiment, the effector protein is selected from the group consisting of: AvrPto, AvrPtoB, and HopAO1. In another non-limiting embodiment, the heterologous protein is a plant protein. In a further non-limiting embodiment, the plant protein is HTA1. In one non-limiting embodiment, the type III secretion system is derived from Pseudomonas syringae spp., Erwinia spp., Xanthomonas spp., Ralstonia spp., Pantoea spp., or Burkholderia spp. In another non-limiting embodiment, the cell further comprises a binary plasmid comprising at least a first nucleic acid sequence flanked by one or more T-DNA border sequence(s). In a further non-limiting embodiment, said first nucleic acid sequence is a sgRNA sequence. In one non-limiting embodiment, the cell is an Agrobacterium cell. In another non-limiting embodiment, the cell is an Ensifer cell.

In another non-limiting embodiment, the present disclosure provides a method for transforming a plant cell comprising: (a) co-culturing at least a first plant cell with the Rhizobium cell of claim 11; and (b) screening for or selecting at least a first plant cell transformed with the nucleic acid sequence comprised in said binary vector. In one non-limiting embodiment, the nucleic acid sequence modifies an agronomic trait. In another non-limiting embodiment, the at least first plant cell is transiently transformed. In yet another non-limiting embodiment, the at least first plant cell is stably transformed. In one non-limiting embodiment, the at least first plant cell is comprised in an explant from a plant seed, seedling, callus, cell suspension, cotyledon, meristem, leaf, root, or stem; and the explant is contacted with the Rhizobium cell. In another non-limiting embodiment, the at least first plant cell is a dicot cell. In a further non-limiting embodiment, the dicot cell is a cotton, soybean, rapeseed, sunflower, tobacco, sugarbeet, or alfalfa cell. In another non-limiting embodiment, the at least first plant cell is a monocot cell. In a further non-limiting embodiment, the monocot cell is a corn, rice, wheat, sorghum, barley, oat, or turfgrass cell. In one non-limiting embodiment, the cell is an Agrobacterium cell. In another non-limiting embodiment, the cell is an Ensifer cell.

In yet another non-limiting embodiment, the present disclosure provides a method of site-specific DNA modification comprising: (a) co-culturing at least a first plant cell with a recombinant Rhizobium cell comprising a bacterial type III secretion system (T3SS), wherein said cell comprises a recombinant DNA construct comprising at least a first nucleic acid sequence operably linked to a T3SS-specific promoter, wherein said first nucleic acid sequence encodes a site-specific DNA modifying enzyme comprising a T3SS signal sequence, wherein the cell further comprises a binary plasmid comprising at least a first nucleic acid sequence flanked by one or more T-DNA border sequence(s); and (b) screening for or selecting at least a first plant cell comprising a modified genome. In one non-limiting embodiment, the site-specific DNA modifying enzyme is selected from the group consisting of: an endonuclease, a recombinase, a transposase, a deaminase, and a helicase. In another non-limiting embodiment, the site-specific DNA modifying enzyme is a CRISPR endonuclease. In a further non-limiting embodiment, the CRISPR endonuclease is in complex with a sgRNA. In yet a further non-limiting embodiment, the sgRNA comprises a copy of a spacer sequence complementary to a protospacer sequence within at least a first DNA target sequence. In one non-limiting embodiment, the DNA modification results in modified expression of the gene of interest in the plant. In another non-limiting embodiment, the modification event results in reduction or elimination of expression of the gene of interest. In another non-limiting embodiment, the modification event results in an increase of expression of the gene of interest. In one non-limiting embodiment, the cell is an Agrobacterium cell. In another non-limiting embodiment, the cell is an Ensifer cell.

In yet another non-limiting embodiment, the present disclosure provides a method of site-specific DNA modification comprising: (a) co-culturing at least a first plant cell with a recombinant Rhizobium cell comprising a bacterial type III secretion system (T3SS), wherein said cell comprises a recombinant DNA construct comprising at least a first nucleic acid sequence operably linked to a T3SS-specific promoter, wherein said first nucleic acid sequence encodes a site-specific DNA modifying enzyme comprising a T3SS signal sequence, wherein the recombinant DNA construct comprises a second nucleic acid sequence operably linked to a T3SS-specific promoter, wherein said second nucleic acid sequence encodes a heterologous protein that functions to suppress innate immunity in plants, and wherein the cell further comprises a binary plasmid comprising at least a first nucleic acid sequence flanked by one or more T-DNA border sequence(s); and (b) screening for or selecting at least a first plant cell comprising a modified genome. In one non-limiting embodiment, the site-specific DNA modifying enzyme is selected from the group consisting of: an endonuclease, a recombinase, a transposase, a deaminase, and a helicase. In another non-limiting embodiment, the site-specific DNA modifying enzyme is a CRISPR endonuclease. In a further non-limiting embodiment, the CRISPR endonuclease is in complex with a sgRNA. In yet a further non-limiting embodiment, the sgRNA comprises a copy of a spacer sequence complementary to a protospacer sequence within at least a first DNA target sequence. In one non-limiting embodiment, the DNA modification results in modified expression of the gene of interest in the plant. In another non-limiting embodiment, the modification event results in reduction or elimination of expression of the gene of interest. In another non-limiting embodiment, the modification event results in an increase of expression of the gene of interest. In one non-limiting embodiment, the cell is an Agrobacterium cell. In another non-limiting embodiment, the cell is an Ensifer cell.

Plant genetic transformation using pathogenic bacteria such as Agrobacterium tumefaciens has been widely utilized deliver genes of interest into a host plant. The molecular basis of genetic transformation of plant cells by Agrobacterium is the transfer of a segment of DNA (T-DNA) located on a large tumor-inducing (Ti) or rhizogenic (Ri) bacterial plasmid from the bacterium and integration of the T-DNA into the plant nuclear genome. To date, considerable effort had been required to apply even well-developed transformation procedures, such as Agrobacterium-mediated transformation, to different plant species. Plants of different species often exhibit substantial physiological differences that impact Agrobacterium-mediated genetic transformation efficiency. Methods for transformation that work for one plant species often do not work effectively, if at all, in other plant species. Thus, the ability to transform a plant species is not necessarily predictive of the ability to transform even related plant species. This is particularly true for bacterial transformation, which involves complex biochemical interactions between the bacterial strains used and target plant cells. Agrobacterium has been the primary bacterial genus used for transferring exogenous DNA into the genomes of plants; however, there exists evidence that other plant-associated bacteria, including (but not limited to) Sinorhizobium/Ensifer and Mesorhizobium, can be modified to mediate gene transfer into various plant species (Broothaerts et al. Nature 433:629-633, 2005).

Many gram-negative bacteria that are pathogenic utilize a complex secretion system called the type III secretion system (T3SS) to deliver proteins directly into their eukaryotic hosts. Many secreted proteins, such as effectors, interfere with the host cellular processes that function in innate immunity and defense responses. These effectors are important for virulence as they function to block detection of microbial molecules, thus allowing the bacteria to thrive in the host environment and cause diseases. The function of the T3SS in pathogenic bacteria have been extensively studied, with most of the T3SS effectors having been identified and their ability to suppress plant immunity or alter plant metabolic functions to cause disease well-understood. Interestingly, although the effector content varies among pathogens, the genes encoding the T3SS are broadly conserved and functional when heterologously expressed in non-pathogenic bacteria such as E. coli and Pseudomonas fluorescens.

Active plant defense against microbial infection relies on innate immune responses triggered by two layers of microbial recognition. The first layer involves the perception of conserved microbial molecules called pathogen-associated molecular patterns or microbe-associated molecular patterns (PAMPs/MAMPs) by pattern recognition receptors (PRRs). This recognition leads to PAMP-triggered immunity (PTI) and is often sufficient to prevent pathogen growth. PAMPs are microbe-derived molecules that are essential for pathogens and are accordingly well-conserved across taxa. Therefore, PTI gives plants a broad-spectrum and durable resistance against non-adapted pathogens. Host-adapted pathogens, however, secrete effectors that block PTI in plants. Over time, plants evolved to form the second layer of defense. This defense is mediated by direct or indirect recognition of specific microbial virulence effectors from host-adapted pathogens through intracellular immune receptors in the host and lead to an effector-triggered immune response which counteracts the pathogen's ability to suppress PTI.

Despite being a gram-negative plant pathogenic bacteria species, Agrobacterium tumefaciens lacks a T3SS and instead relies on the type IV secretion system (T4SS) for its virulence. The T4SS enables transfer of both DNA and proteins directly into host cells (Christie, Biochim. Biophys. Acta, 1694:219-234, 2004). The plant endogenous defense may be a factor in how receptive a plant species is to Agrobacterium-mediated transformation. Arabidopsis thaliana is highly recalcitrant for Agrobacterium-mediated transient transformation. The A. thaliana PRR EF-Tu receptor (EFR) is able to perceive Elongation Factor Thermo-unstable (EF-Tu), one of the most abundant bacterial proteins, and activates a common set of signaling events and defense responses that likely reduces Agrobacterium-induced genetic transformation. This is supported by evidence that A. thaliana efr mutants are more susceptible for Agrobacterium-mediated transformation. Therefore, reducing or dampening plant basal immunity is not only essential for a pathogen to successfully cause disease, but also for improving Agrobacterium-induced transformation in recalcitrant plant species. Many T3SS effectors from plant pathogenic bacteria have the ability to suppress plant defense responses. When the T3SS effector AvrPto, which is known for suppressing plant basal defense (Hann and Rathjen, Plant J. 49:607-618, 2007), was expressed in A. thaliana plants transgenically using a dexamethasone (DEX)-inducible promoter, the plants were more susceptible for Agrobacterium-mediated transient transformation (Tsuda et al., Plant J. 69:713-719, 2012). Transient expression of AvrPto by co-infiltration also improved transient gene expression in Brassica.

Salicylic acid production is triggered as a defense response to Agrobacterium infection in plants. Transgenic A. thaliana plants expressing the NahG gene encoding a salicylic acid hydroxylase from Pseudomonas putida showed increased Agrobacterium-mediated transient transformation and this was likely influenced by a reduction in salicylic acid accumulation in the plants. While these approaches did improve Agrobacterium-mediated transient transformation in some plant species, they also required the production of transgenic plants which is laborious and only improved transient transformation. The present inventors surprisingly found that Agrobacterium-mediated transient and stable transformation was enhanced in plant cells through the use of Agrobacterium tumefaciens engineered to express a heterologous T3SS that delivered the T3SS effector AvrPto to plant cells.

Recently, the bacterial species Ensifer has been shown effectively to transfer DNA into a number of plant species. Ensifer differs from Agrobacterium in that the former is considered to be symbiotic with plants and thus provokes a reduced defense response from treated plants. In view of this, the use of non-pathogenic bacterial species such as Ensifer may increase the range of plant species amenable to genetic modification via transformation.

In one non-limiting embodiment, the present disclosure thus provides a Rhizobium cell, the cell comprising (i) a first nucleic acid comprising a sequence encoding a heterologous type III secretion system; and (ii) a Ti plasmid comprising a second nucleic acid comprising a sequence encoding a heterologous site-specific DNA modifying enzyme. The T3SS is highly conserved in pathogens. The heterologous T3SS contemplated by the present disclosure includes, but is not limited to, those derived from Pseudomonas syringae spp., Erwinia spp., Xanthomonas spp., and Ralstonia spp.

In another non-limiting embodiment of the present disclosure, the Rhizobium cell may further comprise a nucleic acid comprising a sequence encoding a heterologous protein capable of being secreted by the heterologous T3SS. In yet another non-limiting embodiment of the present disclosure, the heterologous protein capable of being secreted by the heterologous T3SS is a plant protein, for example, HTA1. In yet another non-limiting embodiment of the present disclosure, the heterologous protein capable of being secreted by the heterologous T3SS is a type III effector. Examples of such type III effectors are known in the art, for example, AvrB, AvrPphB, AvrPto, AvrPtoB, AvrRpm1, AvrRps4, AvrRpt2, HopA1, HopAF1, HopAI1, HopAO1, HopD1, HopE1, HopF2, HopG1, HopI1, HopM1, HopN1, HopQ1, HopU1, HopW1, HopX1, HopZ1a, and HopZ4 from Pseudomonas syringae; AvrAC, AvrBsTXcv, AvrGf2Xfa, AvrXccBXccB186, XopDXcv, and XopDXccB100 from Xanthomonas spp. (Buttner et al., FEMS Microbiol Rev. 40 (6): 894-937, 2016). In yet a further non-limiting embodiment, the heterologous protein capable of being secreted by the heterologous T3SS is a chimeric protein comprising a type III secretion signal, or fragments thereof, and a polypeptide of interest, which can be efficiently secreted into the cytosols of host cells. The present disclosure overcomes substantial limitations in the art, including limited transformation efficiency, by use of engineered Rhizobia such as Agrobacterium cells expressing a heterologous T3SS capable of introducing a heterologous protein into a plant cell. The present disclosure also provides methods and compositions for site-specific DNA modification utilizing engineered Rhizobia cells, such as Agrobacterium cells expressing a T3SS capable of introducing a heterologous protein into a plant cell.

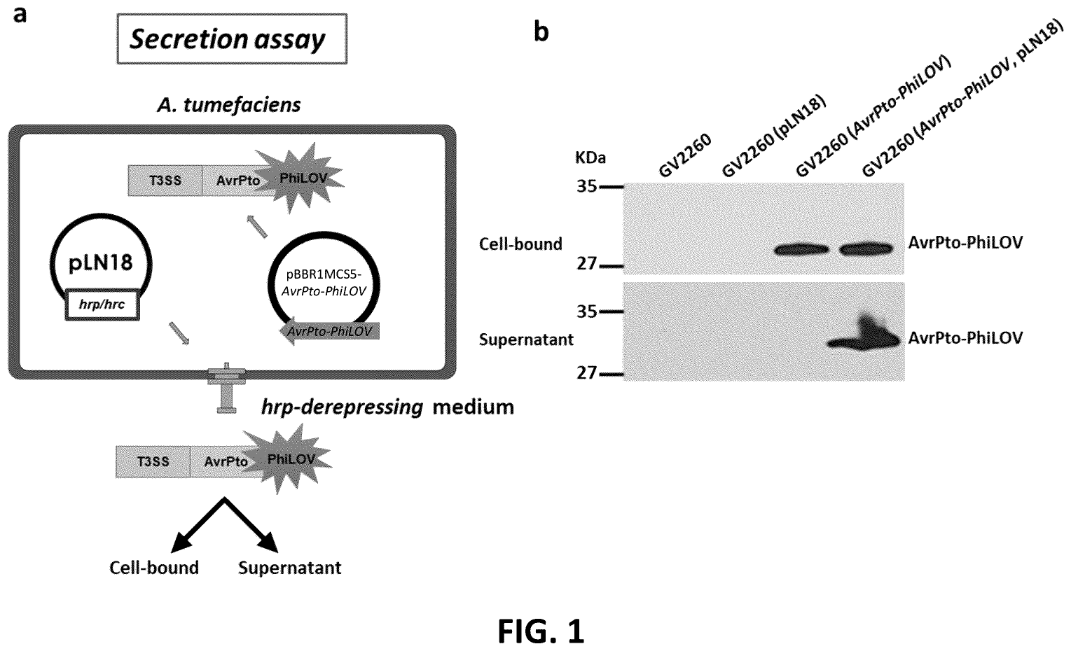

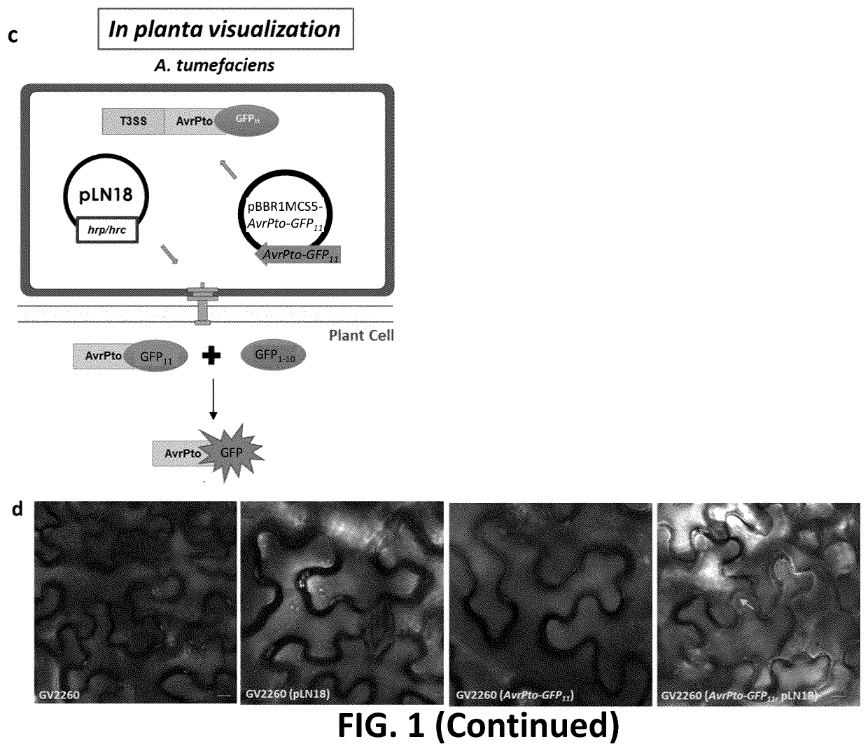

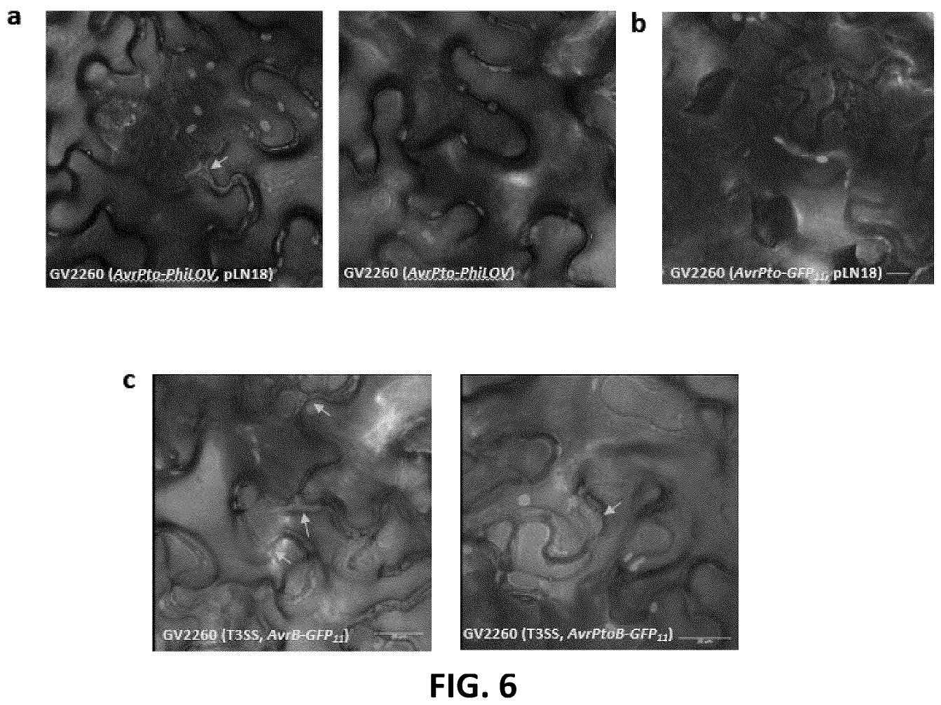

Thus, identifying methods to improve Agrobacterium-mediated transformation in recalcitrant plants as well as increasing transformation efficiencies in general is of great interest. As explained in the working examples below, transformation frequencies of individual transgenic events of as high as 67.5% were obtained for the transformation of wheat (Table 1). These superior results were demonstrated by Agrobacterium expressing a T3SS and a type III effector protein. Furthermore, it is also demonstrated that an Agrobacterium expressing a T3SS can deliver diverse heterologous proteins directly to the plant cell cytosol using the T3SS (FIGS. 1 and 6). Such methods and compositions described herein can be used to deliver DNA modifying enzymes and associated elements directly to host cells for site-specific DNA modification. Direct delivery of DNA modifying enzymes is of value as it may eliminate the need to generate transgenic plants expressing such enzymes.

The present disclosure overcomes limitations in the art by providing, in one non-limiting embodiment, techniques for the use of Rhizobia, such as an Agrobacterium cell comprising a T3SS to improve transformation in important crop plants that were not previously known to be transformable at high efficiencies, including wheat. The present disclosure also provides techniques for the efficient transformation of plants using Rhizobia, such as Agrobacterium, expressing a T3SS, including those already known to be amenable to transformation. The present disclosure also provides methods for Rhizobia-mediated delivery of heterologous proteins directly to the host cytosol, for instance Agrobacterium-mediated delivery. Such delivery, for example, can allow suppression of plant defense systems, the improved integration of T-DNA into the host genome, or site-specific DNA modification through direct delivery of DNA modifying enzymes and associated elements to the host cell.

In another non-limiting embodiment, the present disclosure provides a method of increasing the efficiency of Agrobacterium-mediated gene transfer to a cell. In one non-limiting embodiment, the method comprises the step of introducing into a host cell, a heterologous protein from the Agrobacterium cell into a plant cell during transformation, wherein said introducing is dependent upon the heterologous T3SS. In one non-limiting embodiment of the present disclosure, the heterologous protein may act to modulate the host cell's defense response. In another non-limiting embodiment of the present disclosure, the heterologous protein may act to increase integration of a T-DNA segment into the plant genome. Agrobacterium-mediated transformation methods are well known in the art (e.g., U.S. Pat. No. 5,591,616 and European Patent Application Publication No. EP0672752).

It is well-understood in the art that during Agrobacterium-mediated transformation, DNA is introduced into only a small percentage of target cells. In order to provide an efficient system for identification of cells that have successfully received and integrated the exogenous DNA into their genomes, one may employ a means for selecting those cells that are stably transformed. The DNA that allows for selection or screening may function in a regenerable plant tissue to produce a compound that would confer upon the plant tissue resistance to an otherwise toxic compound. A number of screenable or selectable marker genes are known in the art and can be used in the present disclosure. Examples of selectable markers and genes providing resistance against them are disclosed in Miki and McHugh (J Biotechnol. 107 (3): 193-232, 2004). Genes of interest for use as a selectable, screenable, or scoreable marker would include but are not limited to gus, gfp (green fluorescent protein), luciferase (LUX), genes conferring tolerance to antibiotics like kanamycin (Dekeyser et al., Plant Physiol., 90:217-223, 1989), neomycin, paromomycin, G418, aminoglycosides, spectinomycin, streptomycin, hygromycin B, bleomycin, phleomycin, sulfonamides, streptothricin, chloramphenicol, methotrexate, 2-deoxyglucose, betaine aldehyde, S-aminoethyl L-cysteine, 4-methyltryptophan, D-xylose, D-mannose, benzyladenine-N-3-glucuronidase, genes that encode enzymes that give tolerance to herbicides like glyphosate (e.g. 5-enolpyruvylshikimate-3-phosphate synthase (EPSPS): Della-Cioppa et al., PNAS 83:6873-6877, 1986; U.S. Pat. Nos. 5,627,061; 5,633,435; 6,040,497; and 5,094,945; WO04074443, and WO04009761; glyphosate oxidoreductase (GOX; U.S. Pat. No. 5,463,175); glyphosate decarboxylase (WO05003362 and U.S. Patent Application 20040177399; or glyphosate N-acetyltransferase (GAT): Castle et al. Science, 304:1151-4, 2004., U.S. Patent Publication 20030083480), dalapon (e.g. dehl encoding 2,2-dichloropropionic acid dehalogenase conferring tolerance to 2,2-dichloropropionic acid (Dalapon; WO9927116), bromoxynil (haloarylnitrilase (Bxn) for conferring tolerance to bromoxynil (WO8704181A1; U.S. Pat. No. 4,810,648; WO8900193A)), sulfonyl herbicides (e.g. acetohydroxyacid synthase or acetolactate synthase conferring tolerance to acetolactate synthase inhibitors such as sulfonylurea, imidazolinone, triazolopyrimidine, pyrimidyloxybenzoates, and phthalide; (U.S. Pat. Nos. 6,225,105; 5,767,366, 4,761,373; 5,633,437; 6,613,963; 5,013,659; 5,141,870; 5,378,824; and 5,605,011)); encoding ALS, GST-II), bialaphos or phosphinothricin or derivatives (e.g. phosphinothricin acetyltransferase (bar) conferring tolerance to phosphinothricin or glufosinate (U.S. Pat. Nos. 5,646,024; 5,561,236; 5,276,268; 5,637,489; 5,273,894; and EP 275,957), atrazine (encoding GST-III), dicamba (dicamba monooxygenase (DMO); U.S. Patent Applications 20030115626, 20030135879), or sethoxydim (modified acetyl-coenzyme A carboxylase for conferring tolerance to cyclohexanedione (sethoxydim) and aryloxyphenoxypropionate (haloxyfop) (U.S. Pat. No. 6,414,222)), among others. Other selection procedures can also be implemented including positive selection mechanisms (e.g., use of the manA gene of E. coli, allowing growth in the presence of mannose) and would still fall within the scope of the present disclosure (see also Miki and McHugh, J Biotechnol., 107 (3): 193-232, 2004).

In yet another non-limiting embodiment of the present disclosure, a method for Agrobacterium-dependent site-specific DNA modification is provided. The method, for example, comprises the steps of (a) contacting at least a first plant cell with an Agrobacterium cell, the cell comprising (i) a heterologous T3SS wherein the heterologous T3SS acts to introduce a site-specific DNA modifying enzyme into the at least first plant cell, and (ii) a site-specific DNA modifying enzyme capable of being secreted by the heterologous T3SS; (b) introducing the site-specific DNA modifying enzyme from the cell into the at least first plant cell, wherein said introducing is dependent upon the heterologous T3SS; and (c) binding the site-specific DNA modifying enzyme with at least a first DNA target sequence, wherein the site-specific DNA modifying enzyme mediates a modification event at the at least first DNA target sequence.

As used herein, the term “site-specific DNA modifying enzyme” refers to any enzyme that can modify a nucleotide sequence in a sequence-specific manner. In some non-limiting embodiments, a site-specific genome modification enzyme modifies the genome by inducing a single-strand break. In some non-limiting embodiments, a site-specific genome modification enzyme modifies the genome by inducing a double-strand break. In some non-limiting embodiments, a site-specific genome modification enzyme comprises a cytidine deaminase. In some non-limiting embodiments, a site-specific genome modification enzyme comprises an adenine deaminase. In the present disclosure, site-specific DNA modification enzymes include endonucleases, recombinases, transposases, deaminases, helicases and any combination thereof. In some non-limiting embodiments, the site-specific genome modification enzyme is a sequence-specific nuclease.

In another non-limiting embodiment, site-specific DNA modifying enzyme comprises an endonuclease selected from a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, homologs thereof, or modified versions thereof).

In some non-limiting embodiments, the site-specific DNA modifying enzyme comprises a DNA binding domain operably linked to a deaminase. In some non-limiting embodiments, the site-specific genome modification enzyme further comprises uracil DNA glycosylase (UGI). In some non-limiting embodiments, the deaminase is a cytidine deaminase. In some non-limiting embodiments, the deaminase is an adenine deaminase. In some non-limiting embodiments, the deaminase is an APOBEC deaminase. In some non-limiting embodiments, the deaminase is an activation-induced cytidine deaminase (AID). In some non-limiting embodiments, the DNA binding domain is a zinc-finger DNA-binding domain, a TALE DNA-binding domain, a Cas9 nuclease, a Cas12a nuclease, a catalytically inactive Cas9 nuclease, a catalytically inactive Cas12a nuclease, a Cas9 nickase, or a Cpf1 nickase.

In some non-limiting embodiments, the site-specific DNA modifying enzyme is a recombinase. Non-limiting examples of recombinases include a tyrosine recombinase attached to a DNA recognition motif provided herein is selected from the group consisting of a Cre recombinase, a Gin recombinase, a Flp recombinase, and a Tnp1 recombinase. In a non-limiting embodiment, a Cre recombinase or a Gin recombinase provided herein is tethered to a zinc-finger DNA-binding domain, or a TALE DNA-binding domain, or a Cas9 nuclease. In another non-limiting embodiment, a serine recombinase attached to a DNA recognition motif provided herein is selected from the group consisting of a PhiC31 integrase, an R4 integrase, and a TP-901 integrase. In another non-limiting embodiment, a DNA transposase attached to a DNA binding domain provided herein is selected from the group consisting of a TALE-piggyBac and TALE-Mutator.

Certain non-limiting embodiments of the present disclosure concern the modification of DNA using the CRISPR system. The CRISPR system constitutes an adaptive immune system in prokaryotes that targets endonucleolytic cleavage of the DNA and RNA of invading phage (reviewed in Westra et al., Annu Rev Genet, 46:311-39, 2012). There are three known types of CRISPR systems, Type I, Type II, and Type III. The CRISPR systems rely on small RNAs for sequence-specific detection and targeting of foreign nucleic acids for destruction. The components of the bacterial CRISPR systems are CRISPR-associated (Cas) genes and CRISPR array(s) consisting of genome-target sequences (protospacers) interspersed with short palindromic repeats. Transcription of the protospacer/repeat elements into precursor CRISPR RNA (pre-crRNA) molecules is followed by enzymatic cleavage triggered by hybridization between a trans-acting CRISPR RNA (tracrRNA) molecule and a pre-crRNA palindromic repeat. The resulting crRNA: tracrRNA molecules, consisting of one copy of the spacer and one repeat, complex with a Cas nuclease. The CRISPR/Cas complex is then directed to DNA sequences (protospacer) complementary to the crRNA spacer sequence, where this RNA-Cas protein complex silences the target DNA through enzymatic cleavage of both strands (double-strand break; DSB).

The native bacterial type II CRISPR system requires four molecular components for targeted cleavage of exogenous DNAs: a Cas endonuclease (e.g., Cas9), the house-keeping RNaseIII, CRISPR RNA (crRNA) and trans-acting CRISPR RNA (tracrRNA). The latter two components form a dsRNA complex and bind to Cas9 resulting in an RNA-guided DNA endonuclease complex. For targeted genome modifications in eukaryotes, this system was simplified to two components: the Cas9 endonuclease and a chimeric crRNA-tracrRNA, called guide-RNA (gRNA) or, alternatively, single-guide RNA (sgRNA). Experiments initially conducted in eukaryotic systems determined that the RNaseIII component was not necessary to achieve targeted DNA cleavage. The minimal two component system of Cas9 with the sgRNA, as the only unique component, enables this CRISPR system of targeted genome modification to be more cost effective and flexible than other targeting platforms such as meganucleases, Zn-finger nucleases, or TALE-nucleases which require protein engineering for modification at each targeted DNA site. Additionally, the ease of design and production of sgRNAs provides the CRISPR system with several advantages for application of targeted genome modification. For example, the CRISPR/Cas complex components (Cas endonuclease, sgRNA, and, optionally, exogenous DNA for integration into the genome) designed for one or more genomic target sites can be multiplexed in one transformation, or the introduction of the CRISPR/Cas complex components can be spatially and/or temporally separated.

One non-limiting embodiment of this disclosure is to introduce into a plant cell a sgRNA (single guide RNA), including a copy of a spacer sequence complementary to a protospacer sequence within a genomic target site, and a Cas-associated gene or protein to modify the plant cell in such a way that the plant cell, or a plant comprised of such cells, will subsequently exhibit a DNA modification of interest. The ability to generate such a plant cell derived therefrom depends on introducing Cas-associated proteins and sgRNA of interest using techniques described herein. In one particular (but non-limiting) embodiment of the present disclosure, modification by a DNA modifying enzyme, such as a Cas-associated protein, does not require expression within the host cell. In another non-limiting embodiment, the modification by a DNA modifying enzyme, such as a Cas-associated protein, does not require T-DNA integration into the host genome. Such modification has the advantage of allowing site-specific modification without having to generate plants expressing the CRISPR system.

The expression vector encoding a Cas-associated gene may comprise a promoter. In certain non-limiting embodiments, the promoter is a constitutive promoter, a tissue specific promoter, a developmentally regulated promoter, or a cell cycle regulated promoter. Such a promoter may be bacterial or eukaryotic in origin. Certain contemplated promoters include ones that only express in the germline or reproductive cells, among others. Such developmentally regulated promoters have the advantage of limiting the expression of the CRISPR system to only those cells in which DNA is inherited in subsequent generations. Therefore, a CRISPR-mediated genetic modification (i.e., chromosomal or episomal dsDNA cleavage) may be limited only to cells that are involved in transmitting their genome from one generation to the next. This might be useful if broader expression of the CRISPR system were genotoxic or had other unwanted effects. Examples of such promoters include the promoters of genes encoding DNA ligases, recombinases, replicases, and so on.

Endonucleases are enzymes that cleave the phosphodiester bond within a polynucleotide chain. Examples of endonucleases that cleave only at specific nucleotide sequences are well known in the art and can include, for instance, restriction endonucleases. However, the need for targeted genome engineering as an alternative to classical plant breeding requires highly customizable tools for genome editing. The CRISPR-associated type II prokaryotic adaptive immune system provides such an alternative. A DNA construct that expresses a sgRNA that targets a Cas-associated gene product with endonuclease activity to a specific genomic sequence, such that the specific genomic sequence is cleaved and produces a double-stranded break which is repaired by a double strand break repair pathway, which may include, for example, non-homologous end-joining, homologous recombination, synthesis-dependent strand annealing (SDSA), single-strand annealing (SSA), or a combination thereof thereby disrupting the native locus, may be particularly useful.

In one non-limiting embodiment, a CRISPR system comprises at least one Cas-associated gene encoding a CRISPR endonuclease and one sgRNA comprising a copy of a spacer sequence complementary to a protospacer sequence within an endogenous genomic target site.

In particular (but non-limiting) embodiments, a Cas-associated gene can include any type II CRISPR system endonuclease. Such a Cas-associated gene product would have properties making it amenable to genetic modification such that its nuclease activity and its recognition and binding of crRNA, tra-crRNA, and/or sgRNA could be manipulated.

The present disclosure also provides for use of CRISPR-mediated double-stranded DNA cleavage to genetically alter expression and/or activity of a gene or gene product of interest in a tissue- or cell-type specific manner to improve productivity or provide another beneficial trait, wherein the nucleic acid of interest may be endogenous or transgenic in nature. Thus, in one non-limiting embodiment, a CRISPR system is engineered to mediate disruption at specific sites in a gene of interest. Genes of interest include those for which altered expression level/protein activity is desired. These DNA cleavage events can be either in coding sequences or in regulatory elements within the gene.

This disclosure provides for the introduction of a type II CRISPR system into a cell. Exemplary type II Cas-associated genes include natural and engineered (i.e., modified, including codon-optimized) nucleotide sequences encoding polypeptides with nuclease activity such as Cas9 from Streptococcus pyogenes, Streptococcus thermophilus, or Bradyrhizobium sp.

The catalytically active CRISPR-associate gene (e.g., Cas9 endonuclease) can be introduced into, or produced by, a target cell. Various methods may be used to carry this out, as disclosed herein. The catalytically active CRISPR-associate gene product (e.g., Cas9 endonuclease) can also be introduced directly into a target cell. Various methods may be used to carry this out, as disclosed herein.

In some non-limiting embodiments, the sgRNA and/or Cas-associated gene is transiently introduced into a cell. In certain non-limiting embodiments, the introduced sgRNA and/or Cas-associated gene is provided in sufficient quantity to modify the cell but does not persist after a contemplated period of time has passed or after one or more cell divisions. In such non-limiting embodiments, no further steps are needed to remove or segregate the sgRNA and/or Cas-associated gene from the modified cell. In yet other non-limiting embodiments of this disclosure, double-stranded DNA fragments are also transiently introduced into a cell along with sgRNA and/or Cas-associated gene. In such non-limiting embodiments, the introduced double-stranded DNA fragments are provided in sufficient quantity to modify the cell but do not persist after a contemplated period of time has passed or after one or more cell divisions.

In another non-limiting embodiment, a catalytically active Cas-associated gene product is expressed in a bacterial cell and introduced into a plant cell via a T3SS. In such non-limiting embodiments, the catalytically active Cas-associated gene product is introduced in sufficient quantity to modify the cell (in the presence of at least one sgRNA) but does not persist after a contemplated period of time has passed or after one or more cell divisions. In such non-limiting embodiments, no further steps are needed to remove or segregate the Cas-associated gene product from the modified cell.

In another non-limiting embodiment, a construct that will express a sgRNA and/or Cas-associated gene is created and introduced into a Rhizobia cell, such as an Agrobacterium cell. In other non-limiting embodiments, the cell is a Ensifer cell. In yet another non-limiting embodiment, the vector will produce sufficient quantities of the sgRNAs and/or Cas-associated gene in order for the desired episomal or genomic target site or sites to be effectively modified by CRISPR-mediated cleavage when delivered to a host cell. For instance, the disclosure contemplates preparation of a vector that can be replicated and expressed in a bacterial host, for example, an Agrobacterium cell, wherein the Cas-associated gene product can be delivered to a plant cell by a T3SS.

In other non-limiting embodiments, one or more elements in the vector include a spacer complementary to a protospacer contained within an episomal or genomic target site. This facilitates CRISPR-mediated modification within the expression cassette, enabling removal and/or insertion of elements such as promoters and transgenes.

In another approach, a transient expression vector may be introduced into a cell using a bacterial or viral vector host. For example, Agrobacterium is one such bacterial vector that can be used to introduce a transient expression vector into a host cell. When using a bacterial, viral or other vector host system, the transient expression vector is contained within the host vector system. For example, if the Agrobacterium host system is used, the transient expression cassette would be flanked by one or more T-DNA borders and cloned into a binary vector. Many such vector systems have been identified in the art (Hellens et al., Plant Physiol., 146 (2): 325-332, 2008).

In certain non-limiting embodiments, the CRISPR/Cas9 system can be utilized for targeting insertion of a blunt-end double-stranded DNA fragment into a genomic target site of interest. CRISPR-mediated endonuclease activity can introduce a double stand break (DSB) in the protospacer of the selected genomic target site and microhomology-driven non-homologous end-joining DNA repair inserts the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′ flanking sequence at the cut site of the protospacer in the genomic target site.

In some non-limiting embodiments, genome knowledge is utilized for targeted genetic alteration of a genome. At least one sgRNA can be designed to target at least one region of a genome to disrupt that region from the genome. This non-limiting embodiment of the disclosure may be especially useful for genetic alterations. The resulting plant could have a modified phenotype or other property depending on the gene or genes that have been altered. Previously characterized mutant alleles or introduced transgenes can be targeted for CRISPR-mediated modification, enabling creation of improved mutants or transgenic lines.

In another non-limiting embodiment, a gene targeted for deletion or disruption may be a transgene that was previously introduced into the target plant or cell. This has the advantage of allowing an improved version of a transgene to be introduced or by allowing disruption of a selectable marker encoding sequence. In yet another non-limiting embodiment, a gene targeted for disruption via CRISPR is at least one transgene that was introduced on the same vector or expression cassette as (an) other transgene(s) of interest, and resides at the same locus as another transgene. It is understood by those skilled in the art that this type of CRISPR-mediated modification may result in deletion or insertion of additional sequences. Thus, it may, in certain non-limiting embodiments, be desirable to generate a plurality of plants or cells in which a deletion has occurred, and to screen such plants or cells using standard techniques to identify specific plants or cells that have minimal alterations in their genomes following CRISPR-mediated modification. Such screens may utilize genotypic and/or phenotypic information. In such embodiments, a specific transgene may be disrupted while leaving the remaining transgene(s) intact. This avoids having to create a new transgenic line containing the desired transgenes without the undesired transgene.

In another non-limiting embodiment, the present disclosure includes methods for inserting a DNA fragment of interest into a specific site of a plant's genome, wherein the DNA fragment of interest is from the genome of the plant or is heterologous with respect to the plant. This allows one to select or target a particular region of the genome for nucleic acid (i.e., transgene) stacking (i.e., mega-locus). A targeted region of the genome may thus display linkage of at least one transgene to a haplotype of interest associated with at least one phenotypic trait, and may also result in the development of a linkage block to facilitate transgene stacking and transgenic trait integration, and/or development of a linkage block while also allowing for conventional trait integration.

In another non-limiting embodiment, multiple unique sgRNAs can be used to modify multiple alleles at specific loci within one linkage block contained on one chromosome by making use of knowledge of genomic sequence information and the ability to design custom sgRNAs as described in the art. A sgRNA that is specific for, or can be directed to, a genomic target site that is upstream of the locus containing the non-target allele is designed or engineered as necessary. A second sgRNA that is specific for, or can be directed to, a genomic target site that is downstream of the target locus containing the non-target allele is also designed or engineered. The sgRNAs may be designed such that they complement genomic regions where there is no homology to the non-target locus containing the target allele. Both sgRNAs may be introduced into a cell using one of the methods described above.

The ability to execute targeted integration relies on the action of the sgRNA:Cas-protein complex and the endonuclease activity of the Cas-associated gene product. This advantage provides methods for engineering plants of interest, including a plant or cell, comprising at least one genomic modification.

A custom sgRNA can be utilized in a CRISPR system to generate at least one trait donor to create a custom genomic modification event that is then crossed into at least one second plant of interest, including a plant, wherein CRISPR delivery can be coupled with the sgRNA of interest to be used for genome editing. In other non-limiting embodiments one or more plants of interest are directly transformed with the CRISPR system and at least one double-stranded DNA fragment of interest for directed insertion. It is recognized that this method may be executed in various cell, tissue, and developmental types, including gametes of plants. It is further anticipated that one or more of the elements described herein may be combined with use of promoters specific to particular cells, tissues, plant parts and/or developmental stages, such as a meiosis-specific promoter.

In addition, the targeting of a transgenic element already existing within a genome for deletion or disruption is contemplated. This allows, for instance, an improved version of a transgene to be introduced, or allows selectable marker removal. In yet another non-limiting embodiment, a gene targeted for disruption via CRISPR-mediated cleavage is at least one transgene that was introduced on the same vector or expression cassette as (an) other transgene(s) of interest, and resides at the same locus as another transgene.

Further contemplated are sequential modification of a locus of interest, by two or more sgRNAs and Cas-associated gene(s) according to the disclosure. Genes or other sequences added by the action of such a first CRISPR-mediated genomic modification may be retained, further modified, or removed by the action of a second CRISPR-mediated genomic modification.

“Identity,” as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. Methods to determine “identity” are designed to give the largest match between the sequences tested. Moreover, methods to determine identity are codified in publicly available programs. “Identity” can be readily calculated by methods well-known to those of ordinary skill in the art. Computer programs that can be used to determine “identity” between two sequences may include, but are in no way limited to, GCG (Devereux et al., Nucleic Acids Res. 11:387-395, 1984); the suite of five BLAST programs, three designed for nucleotide sequences queries (BLASTN, BLASTX, and TBLASTX) and two designed for protein sequence queries (BLASTP and TBLASTN) (Altschul et al., J. Mol. Biol. 215:403-410, 1990; Coulson, Trends Biotechnol. 12:76-80, 1994). The BLAST programs are publicly available from NCBI and other sources (NCBI NLM NIH, Bethesda, MD 20894). The well-known Smith Waterman algorithm can also be used to determine identity.

Parameters for polypeptide sequence comparison include the following: Algorithm: Needleman and Wunsch (J. Mol. Biol. 48:443-453, 1970); Comparison matrix: BLOSUM62 from Hentikoff and Hentikoff (PNAS 89:10915-10919, 1992); Gap Penalty: 12; and Gap Length Penalty: 4. A program which can be used with these parameters is publicly available as the “gap” program from Genetics Computer Group, Madison WI. The above parameters along with no penalty for end gap may serve as default parameters for peptide comparisons.

Parameters for nucleic acid sequence comparison are known in the art and may include the following: Algorithm: Needleman and Wunsch (Needleman and Wunsch, Journal of Molecular Biology, 48:443-53, 1970); Comparison matrix: matches=+10; mismatches=0; Gap Penalty: 50; and Gap Length Penalty: 3. A program which can be used with these parameters is publicly available as the “gap” program from Genetics Computer Group, Madison Wis. The above parameters may serve as the default parameters for nucleic acid comparisons.

The nucleic acids provided herein may be from any source, e.g., identified as naturally occurring in a plant or bacterium, or synthesized, e.g., by mutagenesis of a sequence set forth herein.

Coding sequences, such as T3SS coding sequences, type III effector coding sequences, or Cas-associated coding sequences, or portions or complements thereof, may be provided in a recombinant vector or construct operably linked to a heterologous promoter functional in plants or bacteria, in either sense or antisense orientation. In other non-limiting embodiments, plants and plant cells transformed with the sequences may be provided. The construction of vectors which may be employed in conjunction with plant transformation techniques using these or other sequences according to the present disclosure will be known to those of skill of the art in light of the present disclosure (e.g., Sambrook et al., In: Molecular Cloning-A Laboratory Manual (second edition), Cold Spring Harbour Laboratory Press, 1989). The techniques of the present disclosure are thus not limited to any particular nucleic acid sequences. The choice of any additional elements used in conjunction with the coding sequences may depend on the purpose of the transformation. Means for preparing plasmids or vectors containing the desired genetic components are well known in the art. Vectors typically consist of a number of genetic components, including but not limited to regulatory elements such as promoters, leaders, introns, and terminator sequences. Regulatory elements are also referred to as cis- or trans-regulatory elements, depending on the proximity of the element to the sequences or gene(s) they control.

Particularly useful for transformation are expression cassettes. DNA segments used for transforming plant cells will generally comprise the cDNA, gene, or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. In a non-limiting embodiment, introduction of such a construct into a plant may result in increased expression of a particular gene in the plant. In another non-limiting embodiment, introduction of such a construct may result in reduction or elimination of expression of a particular gene.

The use of recombinant DNA molecules for increasing expression of an endogenous gene or overexpressing an exogenous gene in plants is well-known in the art. Exemplary promoters for expression of a nucleic acid sequence include plant promoters such as the CaMV 35S promoter (Odell et al., Nature 313:810-812, 1985), or others such as CaMV 19S (Lawton et al., Plant Mol. Biol. 9:315-324, 1987), nos (Ebert et al., PNAS 84:5745-5749, 1987), Adh (Walker et al., PNAS 84:6624-6628, 1987), sucrose synthase (Yang and Russell, PNAS 87:4144-4148, 1990), α-tubulin, actin (Wang et al., Mol. Cell. Biol. 12:3399-3406, 1992), cab (Sullivan et al., Mol. Gen. Genet., 215:431-440, 1989), PEPCase (Hudspeth and Grula, Plant Mol. Biol. 12:579-589, 1989) or those promoters associated with the R gene complex (Chandler et al., The Plant Cell 1:1175-1183, 1989). Tissue-specific promoters such as leaf-specific promoters, or tissue-selective promoters (e.g., promoters that direct greater expression in leaf primordia than in other tissues), and tissue-specific enhancers (Fromm et al., Nature 319:791-793, 1986) are also contemplated to be useful, as are inducible promoters such as ABA- and turgor-inducible promoters. Any suitable promoters known in the art may be used to express a nucleic acid sequence in accordance with the present disclosure in a plant. In one non-limiting embodiment, such a nucleic acid sequence may encode a DNA sequence that results in increased expression or overexpression of a gene of interest, in a plant.

The DNA sequence between the transcription initiation site and the start of the coding sequence, i.e., the untranslated leader sequence, can also influence gene expression. One may thus wish to employ a particular leader sequence with a transformation construct of the present disclosure. In one non-limiting embodiment, leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences will be known to those of skill in the art in light of the present disclosure.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following examples are included to demonstrate particular (but non-limiting) embodiments of the present disclosure. It should be appreciated by those of skill in the art that the techniques disclosed in the examples which follow represent techniques discovered by the inventors to function well in the practice of the present disclosure, and thus can be considered to constitute particular modes for its practice. However, those of skill in the art should, in light of the present disclosure, appreciate that many changes can be made in the specific embodiments which are disclosed and still obtain a like or similar result without departing from the concept, spirit and scope of the present disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the present disclosure as defined by the appended claims.

### Example 1

### Materials and Methods Used for Experiments

### Plant Materials and Growth Conditions.

For root transformation assays, Arabidopsis thaliana plants were grown in B5 medium in environment-controlled growth chambers at 24° C., 70% humidity, in 16/8 hour light/dark photoperiod with a light intensity of 50-100 μE m⁻² s⁻¹. For leaf infiltration assays, A. thaliana plants were grown in metromix soil in a controlled growth chamber at 22° C., 75% humidity, in 8/16 hour light/dark photoperiod with a light intensity of 140 μE m⁻² s⁻¹ using fluorescent tubes. Nicotiana benthamiana plants were grown in soil in a controlled growth room at 24° C., 75% humidity, in 16/8 hour light/dark photoperiod with light intensity of 140 μE m⁻² s⁻¹ using fluorescent tubes.

### Bacterial Genetic Manipulations and Plasmid Construction.

Promoter sequences of AvrPto (116 bp upstream of the start codon) followed by coding sequences without stop codon (from P. syringae pv. tomato strain DC3000) as well as codon-optimized PhiLOV2.1 sequences were synthesized and cloned into a broad host range vector pBBR1MCS5 at Eco53kl and KpnI site. AvrPto containing its native promoter was amplified and cloned into the XhoI and XbaI sites of pBBR1MCS4 to generate pBBR1MCS4::AvrPto. Coding sequences of AvrPtoB and HopAO1, along with their native promoters (from P. syringae pv. tomato strain DC3000), were synthesized and cloned into Eco53kl and KpnI site of pBBR1MCS5. Promoter sequences used for AvrPtoB and HopAO1 are 93 bp and 86 bp, respectively, upstream of the start codon. AvrRpm1N consists of 199 bp upstream from the start codon and the first 267 bp of the CDS from P. syringae pv. maculicola (Upadhyaya et al., Mol Plant-Microbe Interact. 27:255-264, 2014; Rentel et al., PNAS 105:1091-1096, 2008). AvrRps4N is defined as 129 bp upstream from the start codon and the first 411 bp of the CDS from P. syringae pv. pisi (Sohn et al., Plant Cell 19:4077-4090, 2007; Hinsch and Staskawicz, Mol Plant-Microbe Interact. 9:55-61, 1996). Codon-optimized sequences of full-length and truncated HTA1 (coding first 39 amino acids; Tenea et al., Plant Cell 21:3350-3367, 2009) driven by promoters and N-terminal sequences of AvrRpm1 as well as AvrRps4 were synthesized and cloned into Eco53kl and KpnI site of pBBR1MCS5. All DNA syntheses were carried out at GenScript.

### Bacterial Strains and Growth Conditions.

E coli strain DH5α was used for molecular cloning and was grown at 37° C. in Luria-Bertani (LB) medium. HB101 was used for maintaining pRK2013 helper plasmid. A. tumefaciens strains were grown at 28° C. in YEP agar plates or YEP liquid medium or AB-sucrose medium (Gelvin, Agrobacterium Protocols 77-85, Humana Press, 2006) or hrp-derepressing liquid medium (HDM) (Huynh et al., Science 245:1374-1377, 1989). Antibiotics used were spectinomycin (25 μg ml⁻¹), carbenicillin (10 μg ml⁻¹), rifampicin (10 μg ml⁻¹), kanamycin (50 μg ml⁻¹), gentamycin (25 μg ml⁻¹), or tetracycline (5 μg ml⁻¹). E. coli DH5α competent cells were transformed by standard heat-shock procedure. Electroporation was used to move all the plasmids with the exception of pLN18 to A. tumefaciens strains. Triparental mating was used to mobilize pLN18 to A. tumefaciens strains.

### Immunoblotting

A. tumefaciens strain GV2260 expressing different combinations of AvrPto-PhiLOV and T3SS were grown in YEP agar plates at 28° C. for two days. Two colonies from plates were transferred to liquid hrp-derepressing medium (HDM, pH 6.0) and cultured for 16 hours at 28° C. with shaking at 220 rpm. Pellets from bacterial culture at OD₆₀₀=0.25 were taken for cell-bound fraction analysis. Around 20 ml of the cultures (normalized based on the OD₆₀₀ of the bacterial cultures to get equal protein amount) were separated into pellet and supernatant fractions by centrifuging the culture at 4000 rpm for 15 min at 21° C. Top 15 ml of the supernatant was pipetted out carefully without disturbing the pellet. The supernatant was passed through 0.45 μm Millipore filter, and further centrifugation steps were carried out at 4° C. The supernatant fractions were concentrated by ultra-filtration using Amicon Ultra-15 centrifugal filter (Millipore Corporation), and further concentrated to ^{˜}30 μl using Amicon Ultra-0.5 filters according to manufacturer's instructions. Proteins from cell-bound as well as supernatant fractions were electrophoresed in SDS-PAGE gel and western blot was carried out using PhiLOV-specific antibody.

### Monitoring Effector Delivery Using Confocal Microscopy.

Effector delivery was confirmed by a split GFP system approach that has been described previously (Henry et al., Plant Cell 29:1555-1570, 2017; Park et al., Plant Cell 29:1571-1584, 2017) by the use of GFP₁₁ tagged effectors expressed in bacteria and GFP₁₋₁₀ expressed transiently in N. benthamiana. A. tumefaciens strain GV2260 carrying GFP₁₋₁₀ in a binary vector was infiltrated 24 hours prior to infiltration of GV2260 expressing T3SS, and effectors, as well as negative controls. Recombinant A. tumefaciens GV2260 expressing T3SS and various effectors tagged with GFP₁₁ were grown on YEP plates containing appropriate antibiotics for 2 days at 28° C. Single colonies were inoculated in YEP liquid media for 16 hours, and cells were resuspended in induction media containing 10 mM MES and 200 μM acetosyringone, and incubated at room temperature for 3 hours with slow shaking. OD₆₀₀ of the culture was adjusted to 0.4. Bacterial strains were syringe-infiltrated on the abaxial side of 4-week-old N. benthamiana plants. Forty-eight hours after infiltration, infiltrated area was used for acquiring confocal micrographs with Leica SP8 confocal microscope. GFP was excited at 488 nm and emission was gathered between 493 nm and 550 nm. Chloroplast autofluorescence emission was gathered between 650 nm and 732 nm.

### Transient and Stable Transformation Assays.

N. benthamiana leaf disc assays were carried out as described previously (Anand et al., Mol Plant-Microbe Interact. 20:41-52, 2007). Leaves harvested from greenhouse-grown plants were sterilized using 8% Clorox for 5 minutes and washed 4 times with sterile distilled water. Leaf discs made using a cork borer (0.9 cm) were infected with A. tumefaciens strains for 15 minutes followed by co-cultivation on MS-basal medium for 2 days in the dark at room temperature. Leaf discs were transferred onto MS-basal medium supplemented with cefotaxime (200 mg L⁻¹) and ticarcillin (100 mg L⁻¹). Fifteen days after transfer, fresh and dry weights of 10 discs were measured for leaf disc tumorigenesis assay. For transient transformation assays, leaf discs were collected after 10 days of infection and stained for GUS expression.

Thaliana root transformation assays were performed as described earlier (Gelvin, Agrobacterium Protocols 105-114, Humana Press, 2006). Axenic root segments were infected with A. tumefaciens strain A208 carrying T3SS and T3Es in different combinations, co-cultivated for 48 hours in the dark at room temperature and transferred to a MS basal medium supplemented with cefotaxime (200 mg L⁻¹) and ticarcillin (100 mg L⁻¹). Four weeks after infection, tumor numbers were recorded. For transient transformation assays, A. tumefaciens strain GV3101 carrying different plasmids was used to infiltrate leaves of soil-grown A. thaliana plants. Infiltrated leaves were harvested after 10 days of infection and stained for GUS expression.

### Measuring Plant Defense Genes Expression.

Roots of A. thaliana plants grown on B5 medium for 12 days were infected with Agrobacterium tumorigenic strain A208 expressing AvrPto in combination with and without pLN18. Mock infection and A208 alone were included as negative controls.

Agrobacterium cultures grown overnight in YEP medium at 28° C. were harvested, resuspended in ABM-MS medium containing 200 μM acetosyringone (Wu et al., Plant Methods 10:19, 2014) and incubated at room temperature for 5 hours. The OD₆₀₀ of all A. tumefaciens cultures were adjusted to 1.0 and cultures were pipetted onto roots in a thin layer. For mock infection, ABM-MS medium was used instead of A. tumefaciens cultures. Roots were harvested 2 and 16 hours after infection, rinsed with water, and frozen in liquid nitrogen for RNA extraction. RNA was extracted using RNeasy plant mini kit (Qiagen), and TURBO DNase treatment was done to remove genomic DNA contamination. Reverse transcription reactions were performed with 1 μg of RNA in 20 μl reaction using Oligo (dT) 18 and SuperScript™ III Reverse Transcriptase (Invitrogen). Real-time PCR was done using KiCqStart® SYBR Green qPCR ReadyMix (MilliporeSigma). All samples were repeated three times. Relative expression values were calculated using the 2-\\CT method with UBQ10 as a housekeeping control.

### Bacterial Virulence Genes Expression.

To study the expression levels of Vir genes, recombinant Agrobacterium strains grown in YEP medium overnight at 28° C. were harvested and resuspended in AB induction medium containing 200 μM Acetosyringone (Gelvin, Agrobacterium Protocols 77-85, Humana Press, 2006). After incubation at room temperature with minimal shaking for 8 and 24 hours, bacterial cells were harvested and stored at −80° C. for RNA extraction. RNA was extracted using NucleoSpin® RNA mini kit (Macherey-Nagel) according to manufacturer's instructions, including in-column genomic DNA digestion. cDNA was synthesized using 1.5 μg of RNA using random hexamers (Invitrogen) and Superscript™ III Reverse Transcriptase (Invitrogen). Real-time PCR was done using KiCqStart® SYBR Green qPCR ReadyMix (Millipore Sigma). Minimum three technical replicates and three biological replicates per experiment was done. Relative expression values were calculated using the 2-\\CT method with recA as a housekeeping control.

### Data Analysis.

All data analyses were performed using GraphPad Prism software. The data were analyzed by one-way ANOVA test followed by post-hoc mean separations by Tukey's test. The results were considered to be statistically different when p<0.05.

### Example 2

Secretion Assays to Determine if a Heterologous Type III Secretion System (T3SS) is Functional for Expression, Secretion, and Translocation of Type III Effectors (T3Es) in A. tumefaciens.

A secretion assay was designed to determine if A. tumefaciens comprising a heterologous T3SS is able to express AvrPto and secrete it out into the culture medium (FIG. 1, Panel A). Plasmid pLN18 containing the T3SS-encoding hrp/hrc gene cluster cloned from Pseudomonas syringae pv syringae 61 (Pss61) (Jamir et al., Plant J. 37:554-565, 2004) along with a plasmid containing AvrPto tagged with fluorescent reporter PhiLOV were both transformed into A. tumefaciens strain GV2260. This construct containing the T3SS from Pss61 has been shown to be functional in gram-negative bacteria such as Pseudomonas fluorescens (Upadhyaya et al., Mol Plant-Microbe Interact. 27:255-264, 2014) and Escherichia coli (Huang et al., J Bacteriol., 170:4748-4756, 1988; Gopalan et al., Plant Cell 8:1095-1105, 1996; Pirhonen et al., Mol Plant-Microbe Interact. 9:252-260, 1996). Chromosomal integration of the T3SS from Pss61 is also functional in transforming non-pathogenic P. fluorescens into a pathogen (Thomas et al., Plant J. 60:919-928, 2009).

To monitor the hrp-dependent effector secretion into the medium, recombinant A. tumefaciens strain expressing T3SS and AvrPto-PhiLOV along with appropriate control strains were cultured in hrp-derepressing medium for 16 hours (Huynh et al., Science 245:1374-1377, 1989). Both cell-bound and supernatant fractions were used for western blot (FIG. 1, Panel B). The results showed the presence of AvrPto-PhiLOV in the cell-bound fraction as well as the supernatant fraction for GV2260 containing pLN18 and expressing AvrPto-PhiLOV. A. tumefaciens strain expressing AvrPto-PhiLOV without pLN18 showed AvrPto-PhiLOV only in the cell-bound fraction and not in the supernatant fraction. These results demonstrate that the recombinant A. tumefaciens strain comprising a heterologous T3SS is able to express T3Es and secrete it out into the culture medium.

In planta T3E delivery through T3SS was visualized by using a previously established split GFP system (Henry et al., Plant Cell 29:1555-1570, 2017; Park et al., Plant Cell 29:1571-1584, 2017). GFP₁₁ tagged AvrPto was expressed in A. tumefaciens containing pLN18 and GFP₁₋₁₀ was transiently expressed in N. benthamiana (FIG. 1, Panel C). Four-week-old N. benthamiana leaves were infiltrated with A. tumefaciens that can express GFP₁₋₁₀ within the T-DNA of a binary vector. Twenty-four hours later, the same leaves were re-infiltrated with A. tumefaciens containing pLN18 and expressing AvrPto-GFP₁₁. Forty-eight hours after infiltration, green fluorescence signals were observed inside the plant epidermal cells due to interaction between GFP₁₁ and GFP₁₋₁₀, indicating the delivery of GFP₁₁ into the plant cells. As expected, green fluorescence was not observed in the leaves infiltrated with negative controls (FIG. 1, Panel D). FM4-64 staining of the leaves showed plasma membrane localization of AvrPto-GFP, similar to previous reports (Henry et al., Plant Cell 29:1555-1570, 2017; Park et al., Plant Cell 29:1571-1584, 2017) (FIG. 6, Panel B). A split GFP approach enabled detecting GFP fluorescence in plant cells infiltrated with A. tumefaciens expressing pLN18 and T3Es such as AvrPtoB or AvrB (FIG. 6, Panel C). To further validate the results, PhiLOV-tagged AvrPto was used to monitor AvrPto-PhiLOV delivery (FIG. 6, Panel A). Taken together, the results from immunoblotting and microscopy experiments provide strong evidence that the heterologously expressed T3SS is functional in A. tumefaciens in translocating type III secretion substrates to plant cells.

### Example 3

Type III Effectors Delivered by T3SS in A. tumefaciens Improves Transient and Stable Transformation in N. benthamiana and A. thaliana.

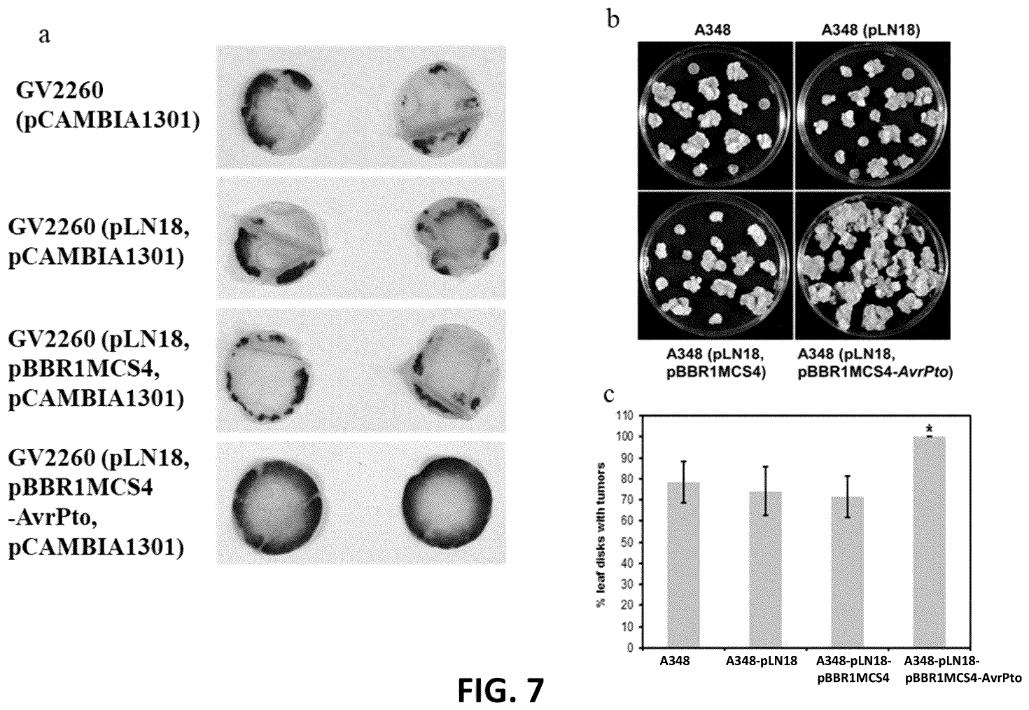

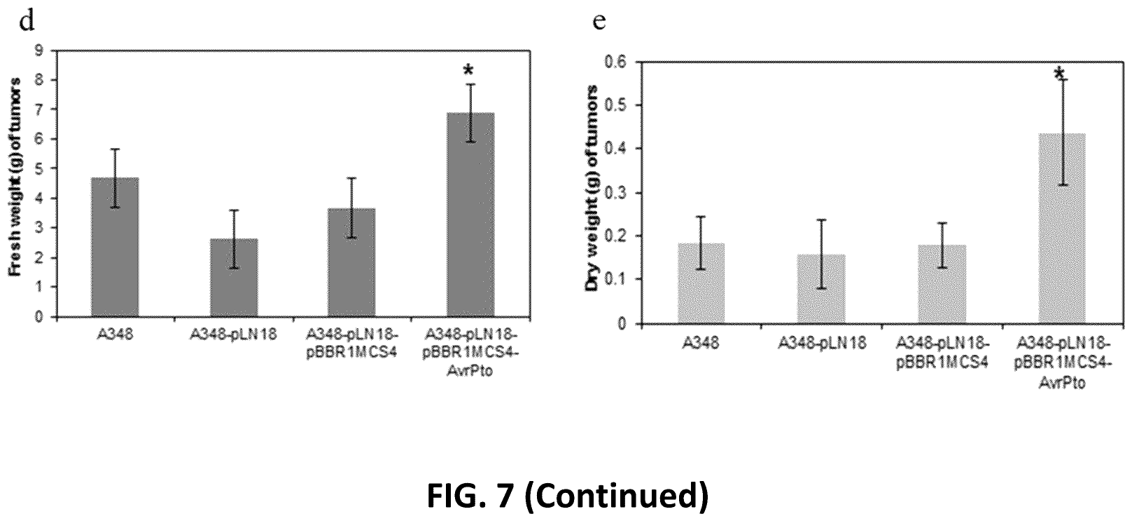

The type III effector AvrPto suppresses plant innate immunity (Xiang et al., Current Biology 18:74-80, 2008), which appears to hinder Agrobacterium-mediated transformation (Lee et al., Plant Cell 21:2948-2962, 2009), and inducible expression of AvrPto in transgenic A. thaliana increases transient transformation efficiency (Tsuda et al., Plant J. 69:713-719, 2012). To determine if AvrPto delivered through T3SS can increase transient transformation, pLN18 (containing T3SS genes) and a plasmid that expresses AvrPto under its native promoter were transferred into a disarmed A. tumefaciens strain GV2260 containing a binary vector that has a β-glucuronidase (GUS) gene within the T-DNA. This engineered A. tumefaciens strain along with appropriate controls was infiltrated into leaves of N. benthamiana plants. As shown in FIG. 7, Panel A, GUS expression was significantly increased when A. tumefaciens expresses T3SS and AvrPto. To determine if T3SS-delivered AvrPto can also increase stable transformation, pLN18 and a plasmid that can express AvrPto were introduced into the tumorigenic strain A348. This engineered A348 strain was used for N. benthamiana leaf disc transformation assay as described (Anand et al., Molecular Plant-Microbe Interactions, 20:41-52, 2007). Leaf discs inoculated with A. tumefaciens strain A348 carrying pLN18 and expressing AvrPto developed significantly more tumors and the tumors were larger in size when compared to controls (FIG. 7, Panels B and C). Significantly, fresh tumor weight was increased between 30% and 60% in comparison with controls, while less variation was found in dry tumor weight, with a 50% increase in leaf discs treated with A348 containing pLN18 and expressing AvrPto than in controls lacking the expression of AvrPto (FIG. 7, Panels D and E).

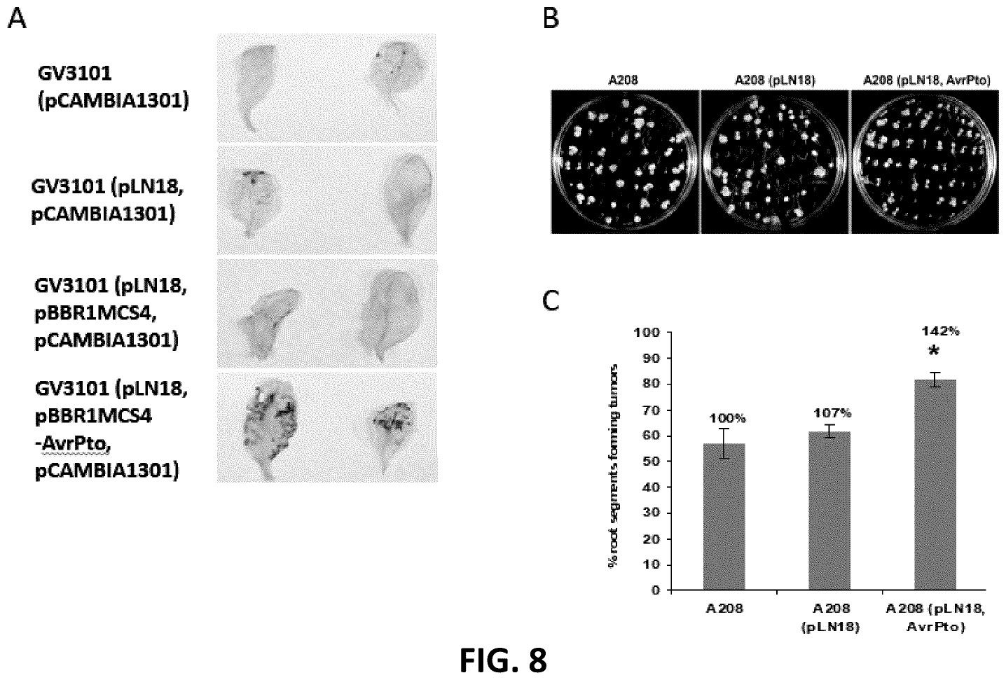

Similar experiments were performed in A. thaliana, using a disarmed strain GV3101 for transient expression and tumorigenic strain A208 for stable root transformation as previously described (Gelvin, Agrobacterium Protocols 105-114, Humana Press, 2006). The results obtained using A. thaliana were similar to those using N. benthamiana wherein expression of T3SS and AvrPto in A. tumefaciens significantly increased both transient and stable transformation (FIG. 8, Panels A and B). Infection with untransformed A. tumefaciens strain A208 or A. tumefaciens strain A208 carrying pLN18 alone caused development of tumors in 55% of the roots (FIG. 8, Panel C). Significantly, infection with A208 carrying both PLN18 and AvrPto increased the development of tumors to 80% (FIG. 8, Panel C). These results are evidence that AvrPto, when delivered through T3SS of engineered A. tumefaciens, can enhance both transient and stable transformation in both N. benthamiana and A. thaliana. In addition to this, the results demonstrate that both T3SS and T4SS are functional in A. tumefaciens.

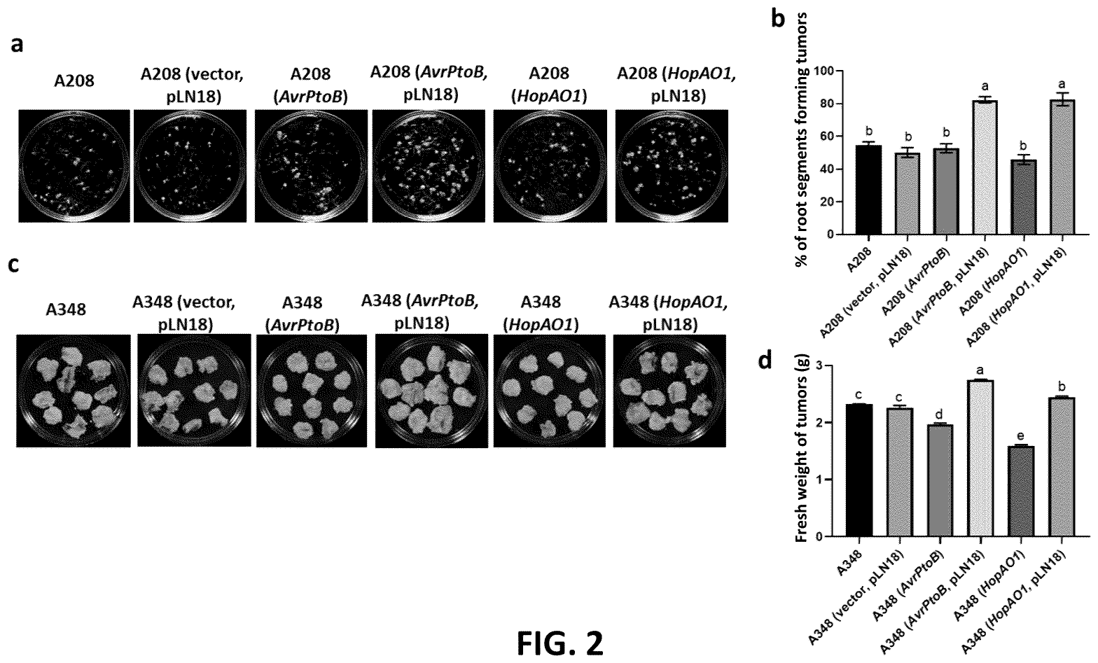

Like AvrPto, several other type III effectors have the ability to suppress plant basal defense to establish/aid growth of pathogens and cause disease (Tang et al., Plant Cell 29:618-637, 2017). To further examine the effect of effectors with plant immune suppression ability on Agrobacterium-mediated plant transformation, two type III effectors from P. syringae pv. tomato, AvrPtoB and HopAO1, were selected. These genes for these two effectors were cloned along with their native promoters in broad host-range vector pBBR1MCS5, and introduced into tumorigenic A. tumefaciens strains A208 and A348 containing PLN18 (T3SS) as described above. Recombinant A. tumefaciens strains were tested for their ability to form tumors on A. thaliana and N. benthamiana using root leaf disc transformation assays. Appropriate negative controls were also included. Similar to AvrPto, both AvrPtoB and HopAO1 significantly increased the percentage of root segments forming tumors and the weight of leaf disc tumors (FIG. 2, Panels A and B). These results showed that both AvrPtoB and HopAO1 can increase stable transformation efficiency in A. thaliana and N. benthamiana when delivered through engineered A. tumefaciens. Furthermore, these results indicate that in addition to AvrPto, several other bacterial effectors that can suppress plant defense can be used to increase Agrobacterium-mediated plant transformation efficiency in plants.

### Example 4

Delivery of Type III Effectors that Suppress Plant Defense Improved Stable Transformation Efficiency in Wheat

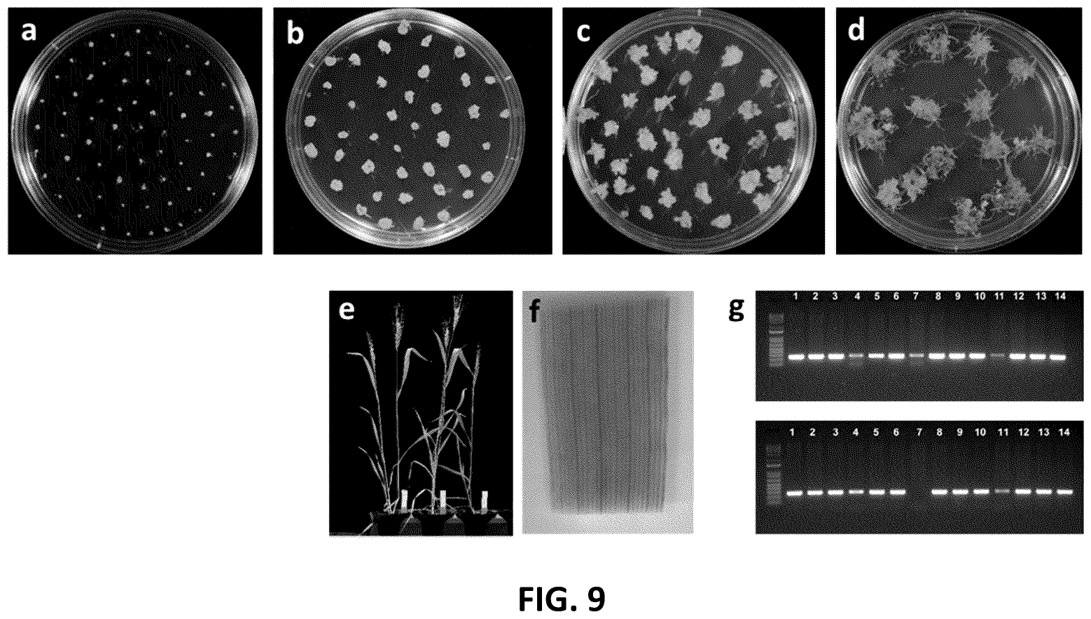

Despite continuous efforts by many groups, efficient and reproducible Agrobacterium-mediated wheat transformation is challenging (Harwood, JXB 63:1791-1798, 2011; Hayta et al., Plant Methods 15:121, 2019). Based on the previous reports, the disarmed A. tumefaciens strain AGL-1 (Hayta et al., Plant Methods 15:121, 2019) and pANIC6B binary vector that carries a kanamycin gene as the bacterial selection marker, a hygromycin gene as a plant selection marker, and a GUS reporter gene were selected. T3SS containing pLN18 and pBBR1MCS5 plasmid with various type III effectors were transferred to A. tumefaciens strain AGL-1 harboring pANIC6B. Immature wheat embryos were infected with this engineered A. tumefaciens strain. As shown in Table 1 below, the number of immature embryos that produced transgenic calli and subsequently regenerated shoots were counted. Plants developed from these regenerated shoots were tested for the presence of reporter genes by GUS histochemical staining as well as PCR analysis of intron-GUS and Hyg genes (FIG. 9). Plants that were positive for at least one of the confirmation tests were considered transgenics.

TABLE 1
Agrobacterium expressing T3SS and AvrPtoB and HopAO1 modulate stable transformation.

| | | | AvrPtoB, | | HopAO1, |
|---|---|---|---|---|---|
| Treatment | Control | AvrPtoB | T3SS | HopAO1 | T3SS |
| No. of embryos used | 40 | 50 | 40 | 44 | 40 |
| No. of embryos developed callus | 22 | 50 | 32 | 37 | 36 |
| No. embryos developed shoots | 14 | 22 | 15 | 5 | 24 |
| No. of embryos developed | 4 | 5 | 5 | 3 | 8 |
| transgenic plants | | | | | |
| Total no. of transgenic plants | 7 | 15 | 17 | 7 | 27 |
| (positive for anyone of the testing) | | | | | |
| % of embryos developed transgenic plants | 10 | 10 | 12.5 | 6.8 | 20 |
| Transformation frequency of individual | 17.5 | 30 | 42.5 | 15.9 | 67.5 |
| transgenic events | | | | | |

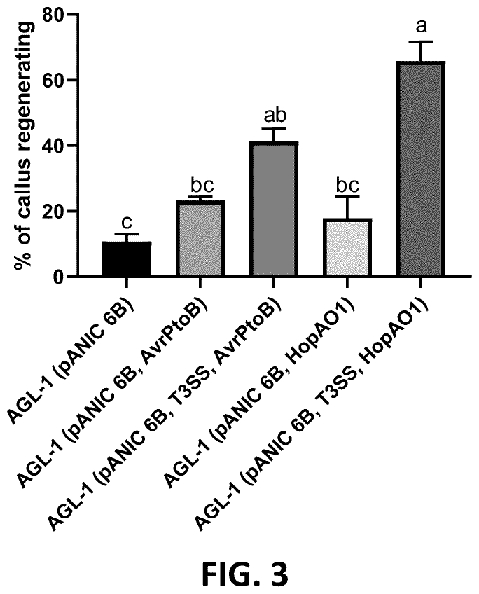

A. tumefaciens strains delivering AvrPtoB or HopAO1 through engineered T3SS significantly increased the percentage of calli that are developed into transgenic shoots (Table 1; FIG. 3). A. tumefaciens strain expressing HopAO1 produced the best results, as the transformation efficiency was ^{˜}400% of initial efficiency. These results indicate that A. tumefaciens with engineered T3SS to deliver type III effectors can be used to increase transformation efficiency of recalcitrant crop species.

### Example 5

### Determination of Virulence of the Recombinant Agrobacterium Strains Expressing AvrPto and T3SS.

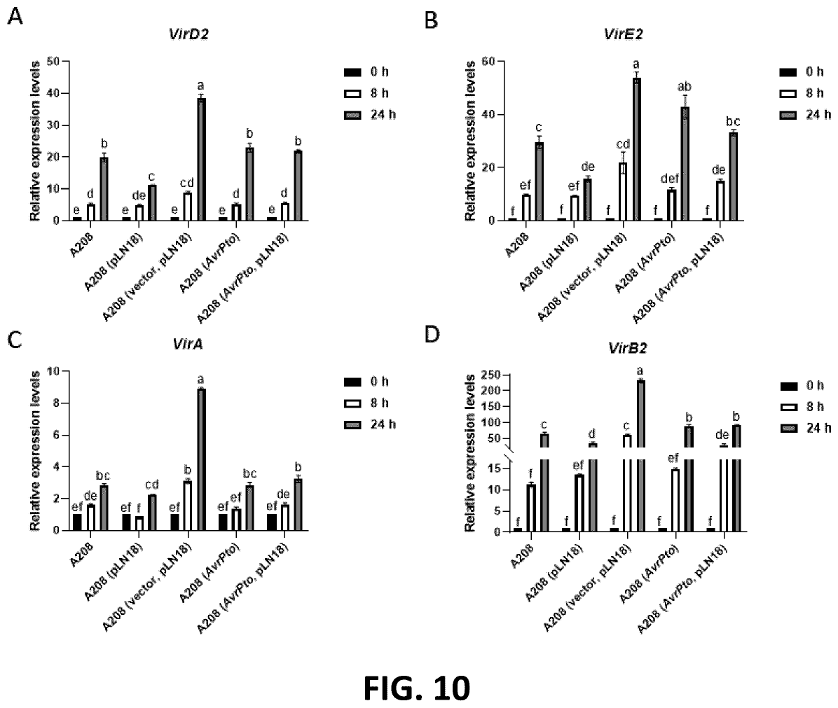

As shown above, A. tumefaciens strains expressing T3SS and type III effectors substantially increased plant transformation efficiency. It is possible that the expression of type III effectors in A. tumefaciens may induce virulence gene (Vir) and thus increase transformation efficiency. To test this, the expression of few selected Vir genes in recombinant A. tumefaciens strains was measured using real-time RT-qPCR. A. tumefaciens strain A208 expressing AvrPto with and without pLN18, along with A208 harboring an empty vector control, were incubated with acetosyringone for Vir gene induction. Bacteria was collected at 0, 8, and 24 hours after incubation and RNA was extracted and checked for Vir gene expression by RT-qPCR. Expression of VirD2, VirE2, VirA, and VirB2, showed significant increase at 8- and 24-hour samples when compared to 0 hours in all tested strains (FIG. 10, Panels A-D, respectively). No significant differences were observed in Vir gene induction between A. tumefaciens strains with or without T3SS and type III effectors. These results, along with the results shown above (FIGS. 2 and 3), indicate that the increase in transformation by A. tumefaciens strains expressing T3SS and type III effectors is not due to increased expression of Vir genes and is most likely due to the delivery of type III effectors into plant cells by engineered A. tumefaciens expressing T3SS.

### Example 6

### Delivery of AvrPto Reduces Plant Defense Genes Expression.

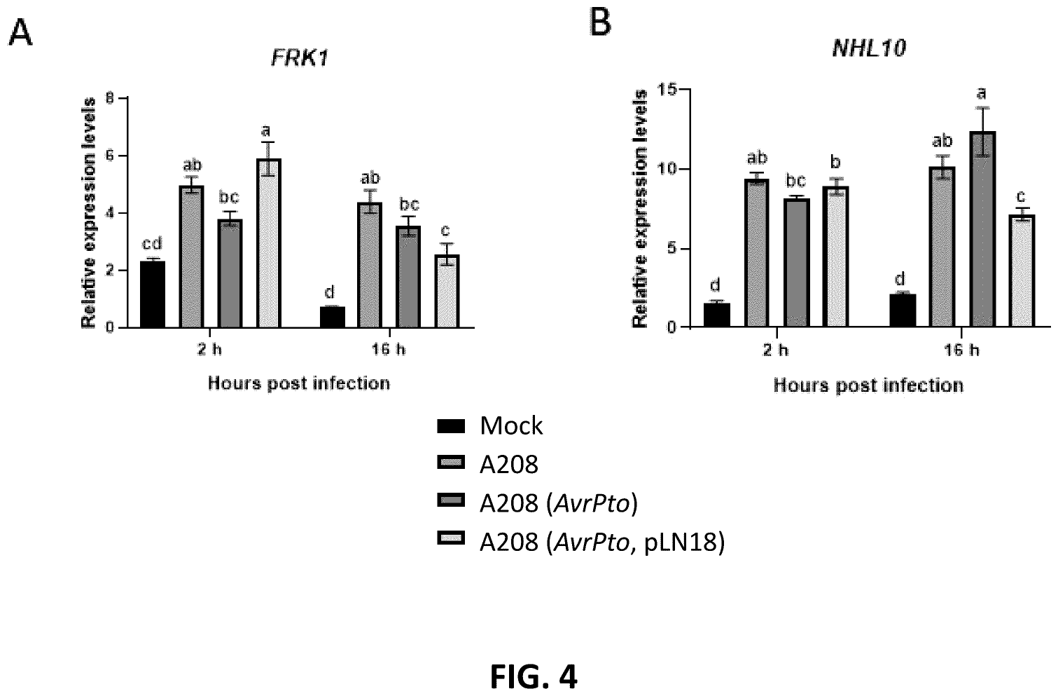

Based on the role of AvrPto in suppressing plant defense, previous reports showing expression of AvrPto in plants increased transient transformation, and the results provided herein showing delivery of AvrPto along with T-DNA into plants increases both transient and stable transformation efficiencies (FIG. 2), it is believed that the increase in plant transformation efficiency that is observed is due to suppression of plant defense responses. To show that AvrPto delivered through engineered A. tumefaciens expressing T3SS is sufficient to suppress plant defense responses, an experiment to measure expression of well-known PTI marker genes, such as FLG22-induced receptor-like kinase 1 (FRK1) and NDR1/HIN1-like 10 (NHL10) was performed by infecting A. thaliana roots with tumorigenic A. tumefaciens strain A208 expressing AvrPto and T3SS. Mock infection and A. tumefaciens expressing AvrPto alone and no AvrPto were used as controls. A. thaliana root tissue was collected at 2 and 16 hours after A. tumefaciens infection. RNA was isolated and to subject to RT-qPCR. Irrespective of the A. tumefaciens strains used, at 2 hours post infection, the FRK1 and NHL10 genes were induced in root samples in response to A. tumefaciens infection when compared to mock control (FIG. 4, Panels A and B). However, at 16 hours post A. tumefaciens infection, transcripts of the FRK1 and NHL10 genes were significantly reduced in root samples infected with A. tumefaciens expressing T3SS and AvrPto when compared to controls (FIG. 4, Panels A and B). These results indicate that AvrPto, when delivered through T3SS expressed by A. tumefaciens, can significantly suppress plant defense response and is therefore contributing to increased plant transformation efficiency.

### Example 7

Delivery of Plant Protein HTA1 from Engineered A. tumefaciens Expressing T3SS Enhances Stable Transformation in N. benthamiana and A. thaliana.

Agrobacterium-mediated transformation is a complex process involving functions of both bacterial virulence proteins and plant proteins (Gelvin, Front Plant Sci. 3:52, 2012; Gelvin, Curr. Opin. Microbiol. 13:53-58, 2010). Histone H2A (HTA1) is one of the first plant proteins identified to be involved in T-DNA integration during Agrobacterium-mediated plant transformation (Mysore et al., PNAS, 97:948-953, 2000). HTA1 overexpression increased Agrobacterium-mediated plant transformation in A. thaliana, rice calli, and Brassica napus (Mysore et al., PNAS, 97:948-953, 2000; Zheng et al., Mol. Plant 2:832-837, 2009; Gelvin, Information Systems Biotechnology News Report, March 2010). Overexpression of full length HTA1, as well as truncated HTA1 (tHTA1; coding only first 39 amino acids), in plants also increases transformation efficiency (Tenea et al., Plant Cell 21:3350-3367, 2009). An experiment was performed to determine if these plant proteins can also be delivered through an engineered A. tumefaciens expressing T3SS to increase plant transformation efficiency.

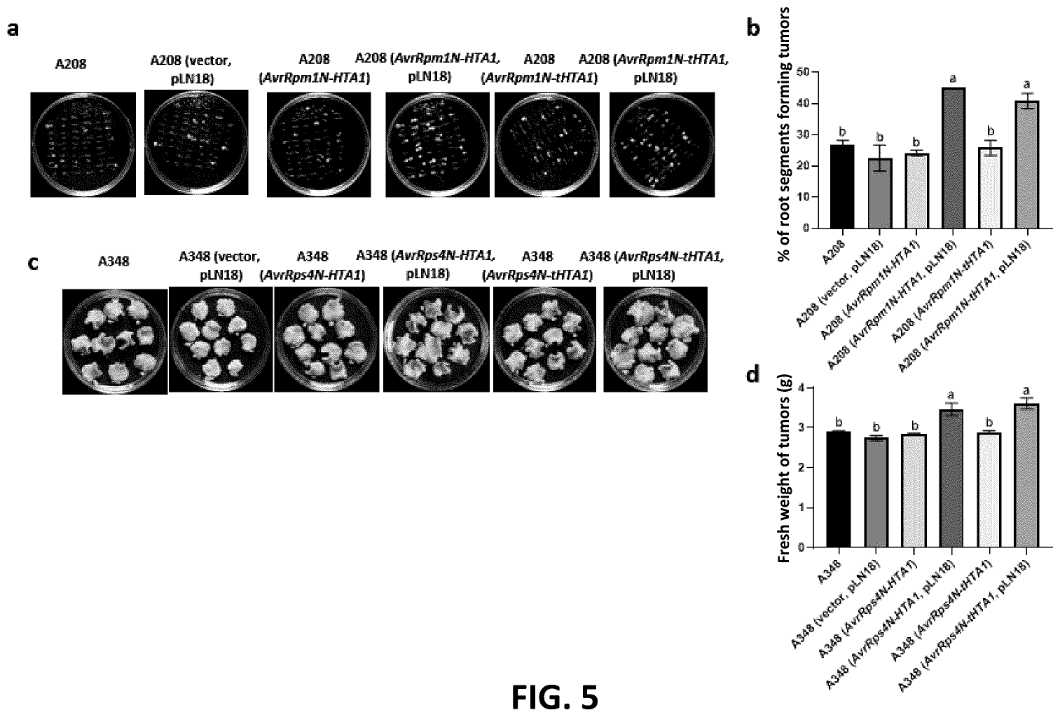

Two different promoters along with N-terminal sequences containing type III signal from bacterial effectors AvrRpm1 and AvrRps4, designated as AvrRpm1N and AvrRps4N respectively, were selected to drive the expression of HTA1 and tHTA1 in A. tumefaciens. Both AvrRpm1N and AvrRps4N previously have been used to express heterologous bacterial and fungal effectors in P. fluorescens for the functional characterization of effectors (Upadhyaya et al., Mol Plant-Microbe Interact. 27:255-264, 2014; Sohn et al., Plant Cell 19:4077-4090, 2007). Codon-optimized HTA1 and tHTA1 driven by AvrRpm1N or AvrRps4N were cloned into pBBR1MCS5 and transferred to tumorigenic A. tumefaciens strains A208 or A348 containing pLN18 (T3SS). Using these A. tumefaciens strains, A. thaliana root and N. benthamiana leaf disc tumor assays were performed as described above along with appropriate controls. FIG. 5 shows that both HTA1 and tHTA1 enhanced stable transformation efficiency in A. thaliana and N. benthamiana. These results indicate that engineered A. tumefaciens expressing T3SS can also be used to deliver plant proteins during transformation.

### Example 8

Delivery of a Heterologous Site-Specific DNA Modifying Enzyme to Plants from Modified Rhizobium Expressing T3SS.

Among the genome editing technologies, CRISPR/Cas9 is emerging as a leading technology to achieve heritable genome modifications in plants because of its simplicity, ease, and high efficiency. The CRISPR/Cas9 system involves recruitment of the Cas9 protein (RNA-dependent DNA endonuclease) to a target site guided by guide RNA (gRNA, 20-nt small RNA complementary to target sequences). There is a great interest in the development of methods to efficiently produce transgene-free genome-edited plants. Such plants are highly desirable in that the Cas9 gene is eliminated from the genome and thus the risk of unintended modifications in untargeted loci is very low. CRISPR/Cas9 mediated genome editing in plants may be performed through either stable or transient transformation. To obtain null segregants from transformed gene edited plants comprising Cas9, progenies must be screened to obtain gene-edited plants where the Cas9 transgene has segregated away by traditional Mendelian segregation. This process can be laborious and time-consuming. Alternatively, transient transformation methods may be used to avoid the insertion of the Cas9 transgene in the plant. This is typically done by transforming protoplasts by PEG treatment or by particle bombardment (Wada et al., BMC Plant Biol. 20:234, 2020). Regeneration of plants from protoplasts, however, is not well developed for many species, especially monocots (Yue et al., Rice 13:9, 2020). This limits the application of transient expression to few species. The engineered Rhizobium expressing a T3SS as described herein may be used for generating transgene-free genome-edited plants of a large number of species in an effective and efficient manner.

SpCas9, which is the Cas9 from Streptococcus pyogenes (SpCas9; 4.10 kbp) was the first Cas9 characterized and has since been used extensively in various organisms for genome editing. Over the years, Cas9 orthologs from other organisms have been identified for their use in genome editing, including SaCas9, which is the Cas9 from Staphylococcus aureus (SaCas9; 3.16 kbp) and CjCas9, which is the Cas9 from Campylobacter jejuni (CjCas9; 2.95 kbp) (Kim et al., Nature Communications 8:14500, 2016; Kaya et al., Plant and Cell Physiology 58:643-649, 2017). SpCas9 and SaCas9 have been successfully used in genome-editing in plants (Wada et al., BMC Plant Biol. 20:234, 2020).

The T3SS of Pss61 that has been described herein has a limitation with regard to the size of the protein that can be secreted by this system. However, there are multiple embodiments of the present disclosure that overcome this limitation. CjCas9 is the smallest Cas9 protein that has been identified thus far. The CjCas9 gene may be cloned into a broad host range vector in the engineered Rhizobium expressing the T3SS of Pss61, expressed in the Rhizobium, and delivered to plants through the Pss61 T3SS. The gRNA is expressed transiently from T-DNA using binary vector in the same engineered Rhizobium that expresses Cas9.

Larger Cas9 proteins may also be utilized in the present disclosure. One non-limiting embodiment utilizes a split Cas9. Split SaCas9 has been optimized for transient expression in tobacco, and requires three different Agrobacterium strains; two strains to express both parts of Cas9 and one to express gRNA (Kaya et al., Plant Cell Physiol. 58:643-649, 2017). Using the present disclosure, the two inactive Cas9 protein fragments are expressed from two different broad host range vectors in two different engineered Rhizobium strains expressing the T3SS of Pss61. The gRNA is expressed transiently from T-DNA using the binary vector in one of the engineered Rhizobium strains that expresses Cas9. Plants are co-infected with the two engineered Rhizobium strains and the Cas9 protein reassembles in the transformed plant. Alternatively, Rhizobium may be engineered to express a T3SS from an organism other than P. syringae pv. syringae. For example, Xanthomonas T3SS is known to deliver bigger effector proteins such as TAL effectors (Rossier et al., 1999). Therefore, Rhizobium may be engineered as described herein to express the Xanthomonas T3SS. The engineered Rhizobium may also comprise a Cas9 gene, such as SaCas9, in a broad host range vector. The gRNA would be expressed transiently from T-DNA using binary vector in the same engineered Rhizobium.

An expression construct containing the Cas9 gene for expression in the engineered Rhizobium will be generated. The promoters and N-terminal signal sequences of AvrRpm1 and AvrRps4, described in Example 1 above, can be utilized. A nuclear localization sequence may also be included. Such examples of a nuclear localization sequence that may be utilized are, for example, a nucleoplasmin nuclear localization signal or the nuclear localization signal of simian virus 40 (SV40) large T antigen (Dingwall et al. JCB 107:841-849, 1988). Other nuclear localization sequences are also known in the art and may be used as described herein. The Cas9 sequence used in the construct should be codon optimized for the Rhizobium species being used for transformation. A terminator sequence may also be included in the construct described to express effectors tagged with GFP₁₁ in P. syringae. An example of such a terminator is the terminator sequence of AvrRpm1 (Park et al., Plant Cell 29:1571-1584, 2017)

### Non-Limiting Illustrative Embodiments of the Inventive Concept(s)

Illustrative embodiment 1. A recombinant Rhizobium cell comprising a bacterial type III secretion system (T3SS), wherein said cell comprises: a recombinant DNA construct comprising at least a first nucleic acid sequence operably linked to a T3SS-specific promoter, wherein said first nucleic acid sequence encodes a site-specific DNA modifying enzyme comprising a T3SS signal sequence.

Illustrative embodiment 2. The Rhizobium cell of illustrative embodiment 1, wherein the site-specific DNA modifying enzyme is selected from the group consisting of an endonuclease, a recombinase, a transposase, a deaminase, a helicase, and any combination thereof.

Illustrative embodiment 3. The Rhizobium cell of illustrative embodiment 1 or 2, wherein the site-specific DNA modifying enzyme is a CRISPR endonuclease.

Illustrative embodiment 4. The Rhizobium cell of illustrative embodiment 3, wherein the site-specific DNA modifying enzyme is selected from the group consisting of SaCas9, SpCas9, StCas9, NmCas9, FnCas9, CjCas9, ScCas9, Cas-CLOVER, xCas9, Cas12a, C2c1, C2c2, Cas13a, Cas12b, Cas14, Cas12k, Cas12e, and any combination thereof.

Illustrative embodiment 5. The Rhizobium cell of any one of illustrative embodiments 1-4, wherein the recombinant DNA construct further comprises a second nucleic acid sequence operably linked to a T3SS-specific promoter, wherein said second nucleic acid sequence encodes a heterologous protein that functions to suppress innate immunity in plants.

Illustrative embodiment 6. The Rhizobium cell of illustrative embodiment 5, wherein the heterologous protein is a bacterial type III effector protein.

Illustrative embodiment 7. The Rhizobium cell of illustrative embodiment 6, wherein the effector protein is selected from the group consisting of AvrPto, AvrPtoB, HopAO1, and any combination thereof.

Illustrative embodiment 8. The Rhizobium cell of illustrative embodiment 5, wherein the heterologous protein is a plant protein.

Illustrative embodiment 9. The Rhizobium cell of illustrative embodiment 8, wherein the plant protein is HTA1.

Illustrative embodiment 10. The Rhizobium cell of any one of illustrative embodiments 1, wherein the type III secretion system is derived from Pseudomonas syringae spp., Erwinia spp., Xanthomonas spp., Ralstonia spp., Pantoea spp., or Burkholderia spp.

Illustrative embodiment 11. The Rhizobium cell of any one of illustrative embodiments 1-10, wherein said cell further comprises a binary plasmid comprising at least one nucleic acid sequence flanked by one or more T-DNA border sequence(s).

Illustrative embodiment 12. The Rhizobium cell of illustrative embodiment 11, wherein the at least one nucleic acid sequence is a sgRNA sequence.

Illustrative embodiment 13. The Rhizobium cell of any one of illustrative embodiments 1-12, wherein the cell is an Agrobacterium cell.

Illustrative embodiment 14. The Rhizobium cell of any one of illustrative embodiments 1-13, wherein the cell is an Ensifer cell.

Illustrative embodiment 15. A method for transforming a plant cell comprising co-culturing at least a first plant cell with the Rhizobium cell of any one of illustrative embodiments 1-10.

Illustrative embodiment 15A. A method for transforming a plant cell comprising: (a) co-culturing at least a first plant cell with the Rhizobium cell of any one of illustrative embodiments 11-14; and (b) screening for or selecting at least a first plant cell transformed with the at least one nucleic acid sequence comprised in said binary plasmid.

Illustrative embodiment 16. The method of illustrative embodiment 15 or 15A, wherein the at least one nucleic acid sequence modifies an agronomic trait.

Illustrative embodiment 17. The method of illustrative embodiment 15, 15A, or 16, wherein the at least first plant cell is transiently transformed.

Illustrative embodiment 18. The method of any one of illustrative embodiments 15-17, wherein the at least first plant cell is stably transformed.

Illustrative embodiment 19. The method of any one of illustrative embodiments 15-18, wherein: the at least first plant cell is comprised in an explant from a plant seed, seedling, callus, cell suspension, cotyledon, meristem, leaf, root, or stem; and the explant is contacted with the Rhizobium cell.

Illustrative embodiment 20. The method of any one of illustrative embodiments 15-19, wherein the at least first plant cell is a dicot cell.

Illustrative embodiment 21. The method of illustrative embodiment 20, wherein the dicot cell is selected from the group consisting of a cotton, soybean, rapeseed, sunflower, tobacco, sugarbeet, or alfalfa cell.

Illustrative embodiment 22. The method of any one of illustrative embodiments 15-21, wherein the at least first plant cell is a monocot cell.

Illustrative embodiment 23. The method of illustrative embodiment 22, wherein the monocot cell is selected from the group consisting of a corn, rice, wheat, sorghum, barley, oat, or turfgrass cell.

Illustrative embodiment 24. The method of any one of illustrative embodiment 15-23, wherein the Rhizobium cell is an Agrobacterium cell.

Illustrative embodiment 25. The method of any one of illustrative embodiments 15-24, wherein the Rhizobium cell is an Ensifer cell.

Illustrative embodiment 26. A method of site-specific DNA modification comprising co-culturing at least a first plant cell with the Rhizobium cell of any one of illustrative embodiments 1-10.

Illustrative embodiment 26A. A method of site-specific DNA modification comprising: (a) co-culturing at least a first plant cell with the Rhizobium cell of any one of illustrative embodiments 11-14; and (b) screening for or selecting at least a first plant cell comprising a modified genome.

Illustrative embodiment 27. The method of illustrative embodiment 26 or 26A, wherein the site-specific DNA modifying enzyme is selected from the group consisting of an endonuclease, a recombinase, a transposase, a deaminase, a helicase, and any combination thereof.

Illustrative embodiment 28. The method of illustrative embodiment 26, 26A, or 27, wherein the site-specific DNA modifying enzyme is a CRISPR endonuclease.

Illustrative embodiment 29. The method of illustrative embodiment 28, wherein the CRISPR endonuclease is in complex with a sgRNA.

Illustrative embodiment 30. The method of illustrative embodiment 29, wherein the sgRNA comprises a copy of a spacer sequence complementary to a protospacer sequence within at least a first DNA target sequence.

Illustrative embodiment 31. The method of any one of illustrative embodiments 26-30, wherein the DNA modification results in modified expression of a gene of interest in the plant.

Illustrative embodiment 32. The method of illustrative embodiment 31, wherein the DNA modification results in reduction or elimination of expression of the gene of interest.

Illustrative embodiment 33. The method of illustrative embodiment 31, wherein the DNA modification results in an increase of expression of the gene of interest.

Illustrative embodiment 34. The method of any one of illustrative embodiments 26-33, wherein the Rhizobium cell is an Agrobacterium cell.

Illustrative embodiment 35. The method of any one of illustrative embodiments 26-34, wherein the Rhizobium cell is an Ensifer cell.

1. A recombinant Rhizobium cell comprising a bacterial type III secretion system (T3SS), wherein said cell comprises:
a recombinant DNA construct comprising at least a first nucleic acid sequence operably linked to a T3SS-specific promoter, wherein said first nucleic acid sequence encodes a site-specific DNA modifying enzyme comprising a T3SS signal sequence.

2. The Rhizobium cell of claim 1, wherein the site-specific DNA modifying enzyme is selected from the group consisting of an endonuclease, a recombinase, a transposase, a deaminase, a helicase, and any combination thereof.

3. The Rhizobium cell of claim 1, wherein the site-specific DNA modifying enzyme is a CRISPR endonuclease.

4. The Rhizobium cell of claim 3, wherein the site-specific DNA modifying enzyme is selected from the group consisting of SaCas9, SpCas9, StCas9, NmCas9, FnCas9, CjCas9, ScCas9, Cas-CLOVER, xCas9, Cas12a, C2c1, C2c2, Cas13a, Cas12b, Cas14, Cas12k, Cas12e, and any combination thereof.

5. The Rhizobium cell of claim 1, wherein the recombinant DNA construct further comprises a second nucleic acid sequence operably linked to a T3SS-specific promoter, wherein said second nucleic acid sequence encodes a heterologous protein that functions to suppress innate immunity in plants.

6. The Rhizobium cell of claim 5, wherein the heterologous protein is a bacterial type III effector protein.

7. The Rhizobium cell of claim 6, wherein the effector protein is selected from the group consisting of AvrPto, AvrPtoB, HopAO1, and any combination thereof.

8. The Rhizobium cell of claim 5, wherein the heterologous protein is a plant protein.

9. The Rhizobium cell of claim 8, wherein the plant protein is HTA1.

10. The Rhizobium cell of claim 1, wherein the type III secretion system is derived from Pseudomonas syringae spp., Erwinia spp., Xanthomonas spp., Ralstonia spp., Pantoea spp., or Burkholderia spp.

11. The Rhizobium cell of claim 1, wherein said cell further comprises a binary plasmid comprising at least one nucleic acid sequence flanked by one or more T-DNA border sequence(s), and wherein the at least one nucleic acid sequence is a sgRNA sequence.

12. (canceled)

13. The Rhizobium cell of claim 1, wherein the cell is an Agrobacterium cell or an Ensifer cell.

14. (canceled)

15. A method for transforming a plant cell comprising:
(a) co-culturing at least a first plant cell with the Rhizobium cell of claim 11; and
(b) screening for or selecting at least a first plant cell transformed with the at least one nucleic acid sequence comprised in said binary plasmid.

16. The method of claim 15, wherein at least one of:
the at least one nucleic acid sequence modifies an agronomic trait;
the at least first plant cell is transiently transformed;
the at least first plant cell is stably transformed;
the at least first plant cell is comprised in an explant from a plant seed, seedling, callus, cell suspension, cotyledon, meristem, leaf, root, or stem; and the explant is contacted with the Rhizobium cell;
the at least first plant cell is a dicot cell selected from the group consisting of a cotton, soybean, rapeseed, sunflower, tobacco, sugarbeet, or alfalfa cell;
the at least first plant cell is a monocot cell selected from the group consisting of a corn, rice, wheat, sorghum, barley, oat, or turfgrass cell;
the Rhizobium cell is an Agrobacterium cell; and/or
the Rhizobium cell is an Ensifer cell.

17-25. (canceled)

26. A method of site-specific DNA modification comprising:
(a) co-culturing at least a first plant cell with the Rhizobium cell of claim 11; and
(b) screening for or selecting at least a first plant cell comprising a modified genome.

27. The method of claim 26, wherein at least one of:
the site-specific DNA modifying enzyme is selected from the group consisting of an endonuclease, a recombinase, a transposase, a deaminase, a helicase, and any combination thereof;
the DNA modification results in modified expression of a gene of interest in the plant;
the Rhizobium cell is an Agrobacterium cell; and/or
the Rhizobium cell is an Ensifer cell.

28. The method of claim 26, wherein the site-specific DNA modifying enzyme is a CRISPR endonuclease, and wherein the CRISPR endonuclease is in complex with a sgRNA.

29. (canceled)

30. The method of claim 28, wherein the sgRNA comprises a copy of a spacer sequence complementary to a protospacer sequence within at least a first DNA target sequence.

31. (canceled)

32. The method of claim 27, wherein the DNA modification results in reduction or elimination of expression of the gene of interest.

33. The method of claim 27, wherein the DNA modification results in an increase of expression of the gene of interest.

34-35. (canceled)
